SEQUENCE LISTING (15 sequences)

SEQ ID NO: 1 - protein, 104 aa, synthetic construct
  REGION 1..104: note = PDB: 5WQ8
KDTTQTKAVYDTNNNFLRNETTTTKGDYTKLASALSSIQGAAVSIAMGDWTALINAVSNDSSSNILSSPSITVMDNGEASFIVGEEVPVITGSTAGSNNDNPFQ

SEQ ID NO: 2 - protein, 104 aa, synthetic construct
  REGION 1..104: note = PDB: 6I1Y
KDKTVTDSRWNSDTDKYEPYSRTEAGDYSTLAAALAGVNGAAMSLVMGDWTALISAVSSDSNSNILSSPSITVMDNGEASFIVGEEVPVITGSTAGSNNDNPFQ

SEQ ID NO: 3 - protein, 101 aa, synthetic construct
  REGION 1..101: note = PDB: 5W68
KPQKGSTVISENGATTINPDTNGDLSTLAQLLSGFSGTAVGVVKGDWMALVQAVKNDSSSNVLSTPSITTLDNQEAFFMVGQDVPVLTGSTVGSNNSNPFN

SEQ ID NO: 4 - protein, 101 aa, synthetic construct; identical to SEQ ID NO: 3
  REGION 1..101: note = PDB: 5ZDH

SEQ ID NO: 5 - protein, 36 aa, synthetic construct
  REGION 1..36: note = isoelectric point determination sequence
KAKITVGEDVPFITGQSQTVGGNVMTMIQRQNVGIT

SEQ ID NO: 6 - protein, 34 aa, synthetic construct
  REGION 1..34: note = cap gate signature sequence
GATGASSLSGSTTGAAGSLGVVSGAAGAASALSG

SEQ ID NO: 7 - protein, 14 aa, synthetic construct
  REGION 1..14: note = central gate signature sequence
QSQTVGGNVMTMIQ

SEQ ID NO: 8 - protein, 23 aa, synthetic construct
  REGION 1..23: note = central gate signature sequence
QTITALTNASQLIGTMAVGPTTT

SEQ ID NO: 9 - protein, 565 aa, synthetic construct
  REGION 1..565: note = amino acid sequence of C6HW33_9BACT
MRQALLSVPTDQHLRTFPLRSRTGRAFWAVLFLLFLVPGPGTVPVAVAAAPGAPPAPTPLALPAPVQKVSMNFRNVDVSVLVRFMSELLQQNIVMDERVKGKLTILSPAEVTVPEAFRIFSDALRMKGFEAVSKKGMIYIVPETQAPPTREMFLYTLENTSAKSIAKTVNAILSKGFAPRPVGGVREGGFDGPIQIVPDKSSNSLIVSATAHDYERLKPILHSLDAQPGEVYVKASLIEISTDKLNNIQVNLLGGVLGSNNSGVVGLTNYGMLGSIVNGATGASSLSGSTTGAAGSLGVVSGAAGAASALSGLNGLVAGVLTGGTFKYNGVNYPNIGELLNALETYSDVRTLSTPEILATDGVKAKITIGEDVPFITGQSQTVGGNVMTMIQRQNVGITLELTPHILSHNRVRLDVKQTISALTNASQVIGTIAVGPTTTKRSAKTNLTISSGQTVVIGGLISKETSINDQGIPFLSDIPILGYLFSNTNNEKKRDDLLIFLTPYVIRNAQDLAVVKHDAPEGLKKFASNNKLLQKLLLDRKTTSTSSDQFLNTVDVPGDPGAPE

SEQ ID NO: 10 - protein, 43 aa, synthetic construct
  REGION 1..43: note = cap gate signature sequence
RTRKEPDDITYRTDAAGQPIYNNNGNRVIASITEGKEIQGDFG

SEQ ID NO: 11 - protein, 23 aa, synthetic construct
  REGION 1..23: note = cap gate signature sequence
GPRNVATVPLGQDLTQPPVAGTG

SEQ ID NO: 12 - protein, 34 aa, synthetic construct
  REGION 1..34: note = cap gate signature sequence
GNIVVDANGNAVTQTTSTQGDFTALASLLGGLNG

SEQ ID NO: 13 - protein, 22 aa, synthetic construct
  REGION 1..22: note = central gate signature sequence
PTITGATASTNNTNPFQTVERK

SEQ ID NO: 14 - protein, 20 aa, synthetic construct
  REGION 1..20: note = central gate signature sequence
QVPILQALAAGNAAFQNVTY

SEQ ID NO: 15 - protein, 22 aa, synthetic construct
  REGION 1..22: note = central gate signature sequence
PILTGTTASAGSSNPATTVDRQ

(19) United States
(12) Patent Application Publication
(10) Pub. No.: US 2024/0125791 A1
(43) Pub. Date: Apr. 18, 2024

(54) SCREENING METHOD FOR AMINO ACID SEQUENCE OF PROTEIN NANOPORE, PROTEIN NANOPORE, AND APPLICATIONS THEREOF

(71) Applicant: SOUTHERN UNIVERSITY OF SCIENCE AND TECHNOLOGY, Guangdong (CN)

(72) Inventors: Yi LI, Guangdong (CN); Ronghui LIU, Guangdong (CN); Yang FU, Guangdong (CN)

(73) Assignee: SOUTHERN UNIVERSITY OF SCIENCE AND TECHNOLOGY, Guangdong (CN)

(21) Appl. No.: 18/399,973

(22) Filed: Dec. 29, 2023

Related U.S. Application Data
(63) Continuation-in-part of application No. PCT/CN2022/099535, filed on Jun. 17, 2022.

(30) Foreign Application Priority Data
Jun. 30, 2021 (CN) 202110739359.0

(51) Int. Cl. G01N 33/68; G01N 33/543

(52) U.S. Cl. CPC G01N 33/6818; G01N 33/54373; G01N 33/6845

(57) ABSTRACT

A screening method for an amino acid sequence of a protein nanopore, a protein nanopore, and applications thereof. The screening method includes: evaluating a characteristic sequence of a dual-pore structure, using a model to search for an amino acid sequence matched with the characteristic feature of the dual-pore structure, removing a redundant candidate sequence and then performing positioning and screening, calculating the matching length and envelope length of the candidate sequence, then performing registration to obtain a relative mismatching relationship with a known protein nanopore, and performing analysis to obtain a final sequence.

### CROSS REFERENCE TO RELATED APPLICATIONS

This application is a continuation-in-part of PCT/CN2022/099535 filed Jun. 17, 2022, which claims priority to the Chinese Patent Application No. CN202110739359.0 filed Jun. 30, 2021, the contents of each of which are incorporated herein by reference in entirety.

### REFERENCE TO AN ELECTRONIC SEQUENCE LISTING

The contents of the electronic sequence listing (SequenceListing.xml; Size: 21 kB; and Date of Creation: Dec. 28, 2023) is herein incorporated by reference in its entirety.

### FIELD

The present disclosure relates to the technical field of nanopore single molecules, and in particular, to a screening method for an amino acid sequence by a protein nanopore, a protein nanopore, and applications thereof.

### BACKGROUND

Accurate detection of biochemical substances at the single-molecule level is a major concern in the fields of medical treatment, hygiene, and environment. However, the conventional substance analysis technology mainly relies on a substance specifically labelled with optical signal for detection, which is not only slow in speed but also expensive. The nanopore single molecule technology is a new detection method developed on the basis of electrophysiology, which requires transportation of substances to be detected through a thin and small nanopore. Since the substances to be detected are different in physicochemical properties, the blocking effects of the substances to be detected on a nanopore current are different when they stay in the pore. Therefore, relevant physicochemical information about the substances to be detected can be obtained by distinguishing blocking currents.

When the substance to be detected has a nucleic acid sequence, the nanopore technique can read sequence information about the single chain of single-molecule nucleic acid in sequence from variation of the current passing through the pore. This method has the advantages of non-labeling, high throughput, low cost, a small amount of samples required, etc. At present, among different means for gene sequencing, the nanopore single-molecule detection as well as substance structure analysis and other aspects thereof have a wide prospect.

Biological nanopore, i.e., porin, has become the main focus in the nanopore single-molecule detection technologies due to its characteristics such as high sensitivity and high reproducibility. Studies have shown that different protein nanopores such as α-hemolysin (α-HL), Mycobacterium smegmatis porin A (MspA), aerolysin, bacteriophage phi29 connector motor protein (phi29 connector), and outer membrane protein (OmpG) and on-membrane channel CsgG of curli biogenesis system can all perform nucleic acid sequence detection, metal ion detection, and analysis of changes in substance configuration and conformation and the like. It should be particularly noted that the protein nanopore has become a main direction of the third-generation sequencing technology for nucleic acid sequencing due to its longer read length. Currently, Oxford nanopore also develops a series of sequencing instruments based on MspA (R7), Lysenin (R8), CsgG (R9), and mutants of CsgG-CsgF, respectively.

At present, commercially stable porins of R9.4.1 version as well as porins of previous versions only have a single read region, and there is in principle the possibility of detection missing for reading long repetitive base sequences. Although protein nanopores disclosed in the prior art can effectively detect a nucleic acid, the detection for repetitive base sequences is carried out with only 4-5 bases, and an error rate thereof is up to 20%. Moreover, it is still challenging to correctly read longer repetitive sequences. In addition, this protein has weak adaptability to solution environments, and has a lot of miscellaneous signals.

Thus, to obtain better a protein nanopore or a substitute thereof would be a long-term research difficulty and technical bottleneck in the art.

### SUMMARY

The present disclosure provides a screening method for an amino acid sequence of a protein nanopore, wherein the screening method includes the following steps in sequence:
(1) acquiring amino acid information about a known protein nanopore, and evaluating a signature sequence of a dual-pore structure by means of a multiple sequence alignment algorithm;
(2) using a hidden Markov model to search for amino acid sequence information matched with the signature sequence of the dual-pore structure, and removing redundant data information;
(3) locating and screening amino acid sequences obtained in step (2) to obtain candidate sequences, and calculating a matching length and an envelope length of the candidate sequences; and
(4) performing registration on the candidate sequences by means of the multiple sequence alignment algorithm, calculating a relative mismatch relationship with the known protein nanopore, and analyzing a structure of the candidate sequences to obtain a final sequence.

In some embodiments, the signature sequence of the dual-pore structure in step (1) is any one of the amino acid sequences represented by protein SEQ ID NO.1˜4.

In some embodiments, conserved match regions used for locating and screening the candidate sequences in step (3) are KDT and LAS.

In some embodiments, the final sequence in step (4) has similarity of 75% or less to the known protein nanopores.

In some embodiments, amino acids screened by the screening method are as shown in the following Table 1:

TABLE 1

| | | | | |
|---|---|---|---|---|
| A0A2R4XIB8_9BORD | A0A2R4XIB8_9BORD | A0A0M0HL95_VIBNE | A0A0B8Q4R6_9VIBR | C9P1Q9_VIBME |
| A0A2J8GRM2_VIBDI | Q87TD7_VIBPA | Q5E1X7_ALIF1 | A0A2N7DHK6_9VIBR | F9RCV9_VIBSN |
| A0A1X1MWB2_9VIBR | A0A1B9QVD0_9VIBR | A0A3A6Q1Q3_9VIBR | A0A2N8ZGP7_9VIBR | A0A1A6KV05_9VIBR |
| A0A0M0HX21_9VIBR | U3BA40_VIBPR | A0A3N9U105_9VIBR | A0A1B1E8H9_VIBNA | A0A1E5DB26_9VIBR |
| Q7MPZ6_VIBVY | A0A233HSR3_9VIBR | F0LRD8_VIBFN | U4KHA5_9VIBR | A0A2M8GU84_9VIBR |
| A0A1R4B794_9VIBR | B7VHF6_VIBTL | A0A090U2E2_9VIBR | A0A090RCJ3_9VIBR | A0A1Y6J271_9VIBR |
| A0A1M5YUW5_9VIBR | E3BIX5_9VIBR | A0A1G8F2U4_9VIBR | A0A2N7D163_9VIBR | U3AQV9_9VIBR |
| A0A427U7Y4_9VIBR | A0A1E5E5Y9_9VIBR | A0A1Q9H0E5_9GAMM | A0A178KPQ7_9GAMM | A0A0J1H2W0_9GAMM |
| A0A0C5WPE0_9GAMM | A0A0J1GNR5_9GAMM | A0A2T3MU32_9GAMM | S3DZY3_9GAMM | A0A0F5V9F4_9GAMM |
| L8J363_9GAMM | A0A1T4UAM9_9GAMM | A0A0F5AR84_9GAMM | D0I313_GRIHO | Q6LLS6_PHOPR |
| A0A1C3EJQ4_9GAMM | D0Z0Q8_PHODD | Q1ZL65_PHOAS | A0A1Y6L124_9GAMM | A0A0F4P8Q5_PSEO7 |
| E1SL34_FERBD | Q3ILP0_PSEHT | Q089U7_SHEFN | Q8EKC9_SHEON | A0A2A6AWN5_9ALTE |
| A0A1H7RDE8_9GAMM | A0A0F4PQ47_9GAMM | A0A0D8CSU2_9GAMM | A0A4R2F307_9GAMM | A0A075P3V8_9ALTE |
| A0A244CPI0_9GAMM | A0A094J9L9_9GAMM | A0A0S2K6C1_9GAMM | A0A1I0E5M3_THASX | A0A0A7EEN0_9GAMM |
| F5ZFZ9_ALTNA | A0A1S2TGE0_9GAMM | U1J8E4_9GAMM | A0A1E7ZDR6_9ALTE | A0A4Q5MF88_9GAMM |
| R9PF56_AGAAL | A8G1I7_SHESH | D4ZEB1_SHEVD | A0A2N0WTU6_9GAMM | Q12T08_SHEDO |
| A1S1X4_SHEAM | A0A1M5Z8V4_9GAMM | K0D8E8_ALTMS | Z5XKA2_9GAMM | B1KN27_SHEWM |
| A0A1S1MMU3_9GAMM | Q47VD4_COLP3 | A0A420E635_9ALTE | A0A0N1EJL4_9GAMM | A0A1M5FAI0_9ALTE |
| A0A2S2DZ48_9ALTE | A0A081KGN5_9GAMM | A0A3S9Q2K7_9GAMM | A0A432W8X7_9GAMM | A0A0F4QDE6_9GAMM |
| A0A1V0KSJ8_9GAMM | A0A161ZS35_9GAMM | R8ASR0_PLESH | A0A269PM82_9GAMM | A0A0Q0IXK5_9GAMM |
| A0A1X7AGD2_9GAMM | A0A1E5IW96_SHECO | A0A411PH35_9GAMM | A0A346NHL1_9ALTE | Q32C46_SHIDS |
| A0A4P6PDB9_9GAMM | L8DF70_9GAMM | A0A135ZZ67_9ALTE | A0A4Q8TT71_9ALTE | A0A4Q9XLM1_9ALTE |
| K6YYR1_9ALTE | A0A3L8PZN5_9GAMM | A8GYT6_SHEPA | A0A090K3H2_9GAMM | A0A1I1QH77_9GAMM |
| A0A1E8FCD0_9ALTE | A9D5J9_9GAMM | A0A1D8S131_9GAMM | A0A432ZR44_9GAMM | K7AJC8_9ALTE |
| A0A1I6GT24_9GAMM | A0A432ZK86_9GAMM | A0A0U2JJS9_9ALTE | F7S076_9GAMM | K7AHG1_9ALTE |
| A0A3E0U7F6_9GAMM | A0A432VQB5_9GAMM | A0A3A6U073_9GAMM | K2L638_9GAMM | A0A4R5H0Q4_9ALTE |
| A0A4P6ARX9_9ALTE | A0A432WGA5_9GAMM | A0A3N5Y227_9ALTE | A0A2S0VWU6_9ALTE | A0A1X7AJ09_9GAMM |
| G4QNE2_GLANF | A0A2K8Y4K2_9ALTE | A0A418YKX3_9GAMM | A0A3D8M5C6_9ALTE | A0A099L6T6_9GAMM |
| A0A2Z6UBN4_GLASK | A0A396U1R7_9GAMM | A0A1G6BG02_9GAMM | A0A432WCL3_9GAMM | K6Y1C2_9ALTE |
| A0A1G8Z1B3_9GAMM | W7R0U8_9ALTE | A0A0W1SUL9_9GAMM | A0A368NNE4_9GAMM | R8AUC3_PLESH |
| A0A094TT15_9GAMM | A0A1Q6CMK1_9GAMM | A0A094JUK1_9GAMM | A0A4P7JLG1_9GAMM | A0A4R4F6P2_9GAMM |
| A0A2S7US16_9GAMM | K6Y484_9ALTE | A0A432ZB03_9GAMM | A0A327XAH8_9GAMM | A0A4P6VYC7_9GAMM |
| A0A4R1KDW2_9GAMM | I1E159_9GAMM | A0A1E7Q245_9GAMM | A0A0S2JJA4_9GAMM | C4K366_HAMD5 |
| A0A1Y6EG42_9GAMM | A0A432X7X6_9GAMM | K2KJQ9_9GAMM | A0A2S0I1A6_9GAMM | A0A0J8GPG7_9ALTE |
| A0A1H4EN06_ALKAM | A3WP11_9GAMM | A0A0M2VC79_9GAMM | A0A432X5D5_9GAMM | H5TAS9_9ALTE |
| I9DQN1_9ALTE | A0A1B7UFI3_9GAMM | F7NYC9_9GAMM | A0A316FZG4_9GAMM | C7R8G0_KANKD |
| A0A0F7M5J4_9GAMM | A0A0F6RB45_9GAMM | A0A2S4HE97_9GAMM | I2JNG5_9GAMM | A0A4R6UUU0_9GAMM |
| A0A1R1M4X8_9GAMM | A0YG33_9GAMM | A0A11IG9N2_9GAMM | A0A419N0Y5_9SPHN | E1VGP8_9GAMM |
| A0A166I3K4_9GAMM | A0A3B3S0V1_9PSED | A0A176H9J8_9GAMM | A0A165RQY1_9GAMM | A0A0C4WQL0_9GAMM |
| A0A1G4TMR5_9SPHN | A0A126R845_9SPHN | A0A1X9NIX2_9GAMM | A0A494T8L5_9SPHN | A0A2X4ADD1_PAULE |
| A0A2N5X781_9GAMM | D4Z3K8_SPHJU | A0A1Z9Z3F0_9GAMM | A0A1H9I7U3_9SPHN | A0A196MU87_9SPHN |
| J2WB20_9SPHN | A0A397PB12_9SPHN | A0A165UCP7_9GAMM | E6WR54_PSEUU | F6EWJ7_SPHCR |
| A0A3M0CWP4_9PROT | B7RSS9_9GAMM | J9A375_9PROT | A0A3F2UZY9_9GAMM | A0A495RUV6_SPHMI |
| A0A173KXX8_9SPHN | A0A1G6UMS9_9SPHN | U3AN14_9CAUL | Q1NDQ6_SPHSS | T0GF70_9SPUN |
| A0A2K9L1D1_9GAMM | A0A0U4VRS9_9PSED | A0A3D9FDZ2_9SPHN | A0A3D96RKX6_9SPBN | A0A0A0F2F8_9GAMM |
| A0A369WLL7_9GAMM | A0A251X7H3_9GAMM | A0A167GJA5_9GAMM | A4A5J7_9GAMM | A0A0E9MNH7_9SPHN |
| A0A3L7E438_9GAMM | A0A437M805_9SPHN | A0A0S2KFE7_9GAMM | I3CIS6_9GAMM | A0A2L0AAA9_9SPHN |
| C6XJ47_HIRBI | A0A437J7N3_9SPHN | A0A0Q5QQ22_9SPHN | A0A372BZH3_9GAMM | A0A1X7H2Z8_9SPHN |
| A0A1H6DFV3_9GAMM | A0A2U2IZM6_9SPHN | A0A37IBG43_9SPHN | A0A059FV20_9RHOB | A0A2U2BSZ9_9RHOB |
| G2IR14_SPHSK | A0A239PP57_9PROT | A0A4E0QTB0_9GAMM | A0A1T4MWA6_9GAMM | W6ME49_9GAMM |
| A0A095X3A9_9GAMM | R9S595_SIMAS | A0A1N6CXJ0_9SPHN | A0A1B6Z9Y9_9SPHN | A0A0H3C3Y1_CAUVN |
| G7UPM4_PSEUP | A0A2P1PW61_9GAMM | A0A2M9EA17_9GAMM | A0A1X9YCA4_9SPHN | A0A061QC35_9PROT |
| A0A0N0K009_9SPHN | M4RVQ2_9SPHN | A0A437GVH6_9SPHN | A0A239JEV9_9SPAN | A0A418WNX9_9SPHN |
| G2FHJ0_9GAMM | A0A1I6JCV2_9SPHN | G4E4N3_9GAMM | Q0A5R3_MARMM | A0A2P7QS74_9SPHN |
| N9UWN8_9SPHN | A0A2N5Y0L6_9GAMM | A0A239L4V7_9SPHN | A0A1E8CG37_9GAMM | A0Z790_9GAMM |
| A0A1B2M327_9GAMM | A0A1B3W8L8_9GAMM | A0A1G5U760_9SPHN | A0A261GSP4_9GAMM | A0A2U9T8L2_9GAMM |
| A0A062VKW4_9RHOB | A0A0Q7R9S3_9CAUL | A0A0Q7THN5_9CADL | A0A1E2VEY3_9GAMM | A0A4R2PTC9_RHOSA |
| A0A1P8X219_9SPHN | A0A0X8R1N8_9SPHN | A0A399RCW4_9RHOB | A0A059F9A5_9RHOB | A0A1Y3CMV6_9GAMM |
| A0A0M5IKW8_SPHS1 | A0A0E2GOQK6_ACIRA | A0A2A3VJG8_9GAMM | A0A420WMB2_9RHOB | Q8P5B6_XANCP |
| E1V5E8_HALED | A0A1Y2Q575_9SPHN | A0A2S8B368_9SPHN | A0A1W7M5B4_9SPHN | A0A3E0H556_9GAMM |
| A0A0Q8QT85_9SPHN | A0A1G6HAY9_9GAMM | A0A0K0XXC2_9GAMM | A0A145WN30_9GAMM | A0A2S7K2Y3_9PROT |
| A0A0R0BKI6_9GAMM | A0A4V3DWS4_9GAMM | A0A4Q1JZM1_9GAMM | A0A3R8Q9B9_9SPHN | A0A1B1YRZ8_9GAMM |
| Q2SDG3_HAHCH | Q1GWD4_SPHAL | A0A0N7GS14_SPHMC | A0A1ZIFC22_9SPHN | A0A0NIC5Q8_9SPHN |
| A0A1I6T6K3_9CAUL | A0A2U3N294_9GAMM | A0A369VU29_9SPHN | A0A2S2FC31_9GAMM | W0ACK8_9SPHN |
| A3UEW2_9RHOB | A0A0N1L4J9_9SPHN | A0A246JI78_9SPHN | A0A257EJC4_9PROT | A0A139BV22_9PROT |
| B4RFI7_PHEZH | B8KQM8_9GAMM | A0A0M4HB54_9GAMM | A0A3M0A8Y7_9GAMM | A0A2S8W2T5_9CAUL |
| F3L1F7_9GAMM | A0A2S5TD46_9GAMM | A0A1H6T2Q8_9SPHN | A0A385BZN2_9GAMM | A0A095B7X9_9SPHN |
| A0A3A3FTQ5_9BURK | A0A1E8DZ41_9GAMM | A0A4R6YN62_9GAMM | A0A4R5U4V7_9GAMM | A0A0Q4FN27_9SPHN |
| D0IZC2_COMT2 | A0A090BUC9_9GAMM | A0A0B8ZKZ8_9SPHN | A0A1V2ERT9_9SPHN | N9BSP8_9GAMM |
| A0A1W2A900_9SPHN | S3MTL6_9GAMM | A0A1L3JA34_9SPHN | A0A1Y0IDQ7_9GAMM | Q2G4Y4_NOVAD |
| A0A0Q5P2J4_9SPHN | A0A3B7PQY8_9GAMM | A0A418WXI5_9BURK | H3NY31_9GAMM | M2U530_9SPHN |
| A0A3P1XIE6_9BURK | A0A4R7DJX1_9SPHN | A0A0Q6L389_9SPHN | A0A410UX45_9BURK | W8X971_CASDE |
| A0A147IV91_9SPHN | A0A0V2FCA0_CAUVI | A0A142LN84_9PROT | A0A399RHB6_9RHOB | N8P3G5_9GAMM |
| A0A37IR866_9PROT | A0A086D155_9GAMM | A0A1H2MHL8_9PSED | A0A4R7PF93_9GAMM | A0A059ZUS1_ACIBA |
| A0A2K8KQJ2_9GAMM | A0A1I5SUB1_9SPEN | A0A3N1POQ6_9GAMM | A0A411HFA8_9GAMM | A0A2A2SKC9_9SPHN |
| A0A1G8AKE2_9RHOO | G0AE23_COLFT | A0A4R7JYY9_9GAMM | A0A1S8CTY8_9GAMM | A0A2A2F225_9GAMM |
| A0A369PSC6_9GAMM | A0A368WYT2_9BURK | A9BXP9_DELAS | A0A4P6X9V1_9SPHN | A0A4Q5VMP5_9GAMM |
| C5TCV4_ACIDE | A0A2T6FHX2_9GAMM | A0A0C1YAC8_9BURK | A0A1U9ND34_9GAMM | A0A0Q9ZWY2_9GAMM |
| A0A1H9APZ5_9GAMM | A0A254TGS9_9BURK | A0A0N0JE75_9PROT | A0A4R2PNS3_9SPAN | A0A0Q4L2F8_9SPHN |
| A0A097EK83_9SPHN | A0A395JKR4_9GAMM | A0A1H9KCM1_9BURK | B3PF18_CELJIJ | A0A1G6VNY9_9GAMM |
| A0A154NED1_9SPHN | A0A3T6E7E2_9RHOB | B8L5U2_9GAMM | I0BMA1_RUBGI | A1TKM7_ACIAC |
| A0A3S2VYR9_9BURK | A0A239CGG1_9BURK | A0A0K8NVX8_IDESA | A0A2S3T626_9BURK | A0A2A4ID89_9SPHN |
| F0KKP0_ACICP | A0A430BJL5_9SPHN | A0A368L123_9BURK | A0A0Q5MKK4_9BURK | A0A1H7GU06_9SPHN |
| A0A1G6TMU7_9BURK | A0A0S9CK81_9SPHN | A0A2S0MXI5_9BURK | A0A238D247_THIOL | A0A1B3PGW3_9BURK |
| A0A2U1C7H7_9BURK | A0A494YDU8_9BURK | A0A2U1CHC9_9BURK | A0A1B4V3U1_9GAMM | A0A1M5E017_9BURK |
| A0A238DWI0_9BURK | A0A3M6Q493_9BURK | A0A2Z6ES36_9BURK | A0A2S9GZ13_9BURK | A0A1T1HJC2_9PSED |
| A0A1X0NEH9_9PSED | A0A0S1XVX2_9BORD | A0A432VLM6_9SPHN | C5A834_BURGB | A0A1I1R3B0_9BURK |
| A0A0L6TG09_9BURK | A0A225LVV7_9BURK | Q8XUS1_RALSO | A0A328ZJ26_9BURK | E0TBQ0_PARBH |
| A0A069PIT2_9BURK | A0A0C1Z0Y4_9BURK | A1WNP1_VEREI | A0A1T0CCW3_9GAMM | A0A0T0PV29_9SPHN |
| A0A4R2N4C1_9BURK | H5WHD3_9BURK | A0A2T6F7R2_9BURK | A0A2P1NNY3_9BURK | A0A2S0MJE8_9BURK |
| A0A221KIU3_VITFI | A0A369AJE4_9BURK | Q1LHW2_CUPMC | A0A1P8FK66_9PROT | A0A2U8GKK5_9RHOO |
| A0A4Q9GX70_9BURK | A0A1JI7Y8Z3_9SPHN | A0A2A2AED5_9BURK | A1W3T7_ACISJ | Q0K5W9_CUPNH |
| A0A1D9H300_9BURK | G4MJT4_9BURK | N6YRW5_9RHOO | L9PM96_9BURK | A0A0D7K9L6_9BURK |
| A0A4Q5V8V9_9GAMM | A0A0H4VYT7_9BORD | A0A1Y0ESE5_9BURK | A0A1I7JC14_9BURK | A0A316EL55_9BURK |
| F2LH25_BURGS | A0A0M2WWU6_9BURK | Q46W94_CUPPJ | A0A0H3KB12_BURM1 | Q5P193_AROAE |
| A0A4U8V7X9_9BURK | E5AKF9_PARRH | A0A0B1Y375_9RALS | A0A0J1CWB5_9BURK | J1EGV9_9BURK |
| A4J9Z0_BURVG | A0A315E6W4_9BURK | A0A1I8DU30_9BURK | A0A0L0MFJ4_9BURK | A0A315AF78_9BURK |
| A0A1N7S437_9BURK | A0A1L6HS93_9BURK | A0A0L6VVR5_9BURK | A0A3N8LD73_9BURK | A0A2N7X874_9BURK |
| A0A1A6DU88_9BURK | A0A158J5H3_9BURK | A0A494XU39_9BURK | A0A1E7X632_9BURK | W0VA07_9BURK |
| F3KWC1_9BURK | A0A346BGJ3_9BURK | N6YLK7_9RHOO | A0A0U3NA82_9BURK | A0A2S7JWX4_9BURK |
| B2JXL4_PARP8 | A0A3N8KT41_9BURK | A0A2S9K9T0_9BURK | A0A0J7JRJ5_9BURK | A0A0A6QG77_9BURK |
| A0A0N0XI54_9NEIS | A0A0Q8RI17_9BURK | A0A208XWU4_9BURK | A0A255ZJW0_9BURK | A0A063BN64_9BURK |
| A0A4P8IWQ8_9BURK | A0A1H8PS83_9BURK | V2JDP8_9BURK | Q63Z30_BURPS | A0A0L1L1D3_9BURK |
| C4K9Q5_THASP | A0A480B3T7_9BURK | A0A4R6QID2_9BURK | F4G6Z8_ALIDK | A0A4R5MBS0_9BURK |
| A0A1G6U2B2_9BURK | A0A062U1O5_9RHOB | A0A1I4K711_9GAMM | A0A4R6EDK3_9RHOO | A0A2N7VP79_9BURK |
| A0A098UCP7_9BURK | A0A3P4AZ46_9BURK | A0A1P8J559_9BURK | U5NC55_9BURK | A0A1H0WN38_9BURK |
| A0A372K515_9BURK | A0A0P0M763_9BURK | A0A1X7K9I0_9BURK | A4BES0_9GAMM | A0A1N6GLF9_9BURK |
| A0A1H2PQ64_9BURK | F5V0R0_RAMTT | A0A1I7LKI2_9BURK | A0A494XBI7_9BURK | A0A345P7W7_9GAMM |
| A0A149VTF1_9PROT | A0A0J1CRE9_9BURK | A0A2N7VZZ6_9BURK | A0A063BJ98_9BURK | A0A1N7RSR5_9BURK |
| A0A318H1P4_9BURK | A0A1S9A9S8_9BURK | A0A1A5X6X5_9BURK | N6XFJ0_9RHOO | A0A059KHTY7_9BURK |
| E1T3W0_BURSG | A0A069IF75_9BURK | A0A4P7R990_9BURK | A0A221A6N6_9BURK | A0A1V4CCD5_9BURK |
| A0A0Q6VKG2_9BURK | A0A1X0XW78_9DELT | A0A257FTP0_9BURK | A0A167HPQ8_9BURK | A0A149VYN0_9PROT |
| A0A2U2I5A5_9BURK | A0A1X7DPD1_TRICW | K9DGT6_9BURK | A0A1Q8IRV7_9BURK | A0A1H0YRT4_9BURK |
| A0A3N7C8J2_9BURK | A0A254NE78_9BURK | A0A0Q7SZY5_9BURK | A0A2J6MQ90_9BURK | A0A4Q7VZU7_9BURK |
| A0A1J0WCN4_9RHOB | A0A437LLM1_9BURK | A0A1V3S732_9BURK | A0A0FSK3C7_9BURK | A0A2R8BPH0_9RHOB |
| A0A1W6MXC1_9RHIZ | A0A143B4V0_9DELT | A0A4V2VPH8_PELSC | D4H3X7_DENA2 | A0A0C2HST3_9DELT |
| A2SKK0_METPP | A0A410K1G8_9BACT | A0A257CM12_9BURK | B9TP47_RICCO | G9PZL4_9BACT |
| A0A1Y4GE63_9BACT | A0A2K8U2X0_9GAMM | C6BTY5_DESAD | A0A073IT15_9BACT | A0A1N6J1Y5_9BRAD |
| A0A317ZCS2_9BACT | A0A151CHF1_9PROT | A0A2D3WF25_9PROT | D5EMQ7_CORAD | A0A290QKU8_9BACT |
| H0PWI7_9RHOO | S6AJF6_SULDS | A0A238D2G7_THIDL | J9ZAN5_LEPFM | A0A3N4URG9_9BURK |
| A0A0R0CKC0_9GAMM | A0A162YZ14_9BURK | B9XBV8_PEDPL | A0A0K8NXZ4_IDESA | F9ZTP9_ACICS |
| A0A2A2AJ90_9BURK | A0A1H0U0D9_9BURK | A0A109BVG9_9BACT | A0A1P8K005_9BURK | A0A1V3S7B9_9BURK |
| A0A411X8H0_9BURK | A0A1I6KEU3_9BURK | A0A4R3K4X1_9FIRM | A0A1Y4DH20_9BACT | B2KC06_ELUMP |
| J0UE18_9BURK | A0A127JUU8_9BURK | A4G6F2_HERAR | A0A1W6L5F4_9BURK | A0A363RKY8_9BURK |
| A0A0U3MZR6_9BURK | A0A217N790_9NEIS | F1VYM7_9BURK | A0A3P3VMG0_9GAMM | A0A1B3LKM2_9PROT |
| A0A4Q6B9C2_9PROT | A0A0G3BE06_9BURK | I0HMC6_RUBGI | F5RCD9_METUF | A0A0M2WTW8_9BURK |
| A0A0Q4YLY7_9BURK | A0A401FQC8_9DELT | A0A4V2FSP9_9BURK | U5NBM0_9BURK | A0A0S4K799_9BURK |
| S7KJ89_9CHLA | Q12FF1_POLS1 | A0A2R4CI38_9BURK | A0A0K1K0C7_9BURK | A0A238DJ48_9BURK |
| A0A2S5T5J2_9BURK | A0A098U3J5_9BURK | A0A0Q6XX65_9BURK | A0A1P8KD37_9BURK | A0A1V4CDF2_9BURK |
| A0A2G8SYW6_9BURK | A0A4P7RE37_9BURK | A0A318H1V0_9BURK | A0A316EMH3_9BURK | A0A4Q9H3X3_9BURK |
| A0A1M4SQX0_9BURK | A1KBQ6_AZOGB | A0A1J4T5T6_9GAMM | A9BXD0_DELAS | A0A4R3V3T1_9PROT |
| A2SKH3_METPP | A0A1E7X722_9BURK | A0A1S9A8Q2_9BURK | A0A244EF87_9BURK | A0A1Y6CEG9_9PROT |
| A0A0A0DT76_9BURK | A0A480AYY7_9BURK | A0A0M2WQ85_9BURK | A0A0Q6W0H9_9BURK | A0A0H2MLC4_VARPD |
| A0A1I7KEB3_9BURK | A0A2S9H4C7_9BURK | A0A1W6LIZ5_9BACT | A0A1M5UET1_9BURK | A0A0L6T9X7_9BURK |
| F8KX56_PARAV | A0A0S9NT86_9BURK | A0A1Y0NFG2_9BURK | A0A0C2BQF3_9BURK | S6APC6_SULDS |
| Q9PGC9_XYLFA | A0A086WE27_9BURK | A0A0U3EE68_9BURK | A0A0Q5H5V3_9BURK | A0A418XQS9_9BURK |
| A0A3A3G1K8_9BURK | A0A0N1AHX9_9PROT | A0A4R5W1R9_9BURK | A0A4Q7LG60_9BURK | B1Y305_LEPCP |
| Q3SKQ8_THIDA | A0A1C31KD9_XANCT | A0A255ZF51_9BURK | A0A1H8B5F3_9BURK | W0VC85_9BURK |
| A0A0H5F3F3_9BACT | A0A090CXX3_9BACT | A0A1G8PLQ8_9BURK | A0A2S7JY50_9BURK | A0A318J3Y4_9BURK |
| J3DBL3_9BURK | A0A1I5S3A2_9BURK | A0A1H8DH90_9BURK | A0A2R4CGM3_9BURK | A0A0U1Q1K2_9BURK |
| A0A3M6QCX8_9BURK | A0A059KM71_9BURK | A0A2N4XU87_9RHOO | A0A3S2OT00_9BURK | MINZY7_DESSD |
| A0A221KAR9_VITFJ | A0A1H7SCK1_9GAMM | A0A1H8Q344_9BURK | A0A3N7HVQO_9BURK | A0A0Q6WHM4_9BURK |
| A0A146GC05_TERSA | H5WJ69_9BURK | F5XY88_RAMTT | A0A4R6RNI8_9BURK | A0A3M6QUN0_9BURK |
| C7RPD0_ACCPU | A0A127ENL2_9RHIZ | A0A1T4WA02_9DELT | A0A2S0MIN7_9BURK | A0A1I1FQE0_9BURK |
| A0A3R8NQG7_9BURK | A0A1N6HLR4_9PROT | A0A239IFN3_9BURK | A0A2M6VKL0_9BURK | A0A4R3LJJ2_9BURK |
| D2U987_XANAP | A0A4U8VCK1_9BURK | L9PKT2_9BURK | A0A1S8FGV2_9BURK | A0A316EW42_9BURK |
| A0A0Q8QH29_9BURK | M2T910_9SPHN | A0A3P1XFL2_9BURK | A0A0Q7T139_9BURK | Q21UB9_RHOFT |
| A0A426VEJ8_9BURK | A0A0Q6LIQ9_9BURK | A0A437NR73_9RHIZ | A0A0Q7AI74_9BURK | D8ITM3_HERSS |
| G8QKY8_AZOOF | A0A4Q8LLL4_9GAMM | A0A0J1D9D4_9BURK | A0A2G8SZ84_9BURK | A0A1H7UF66_9DELT |
| A0A323V4S6_9RHOO | A0A2G8TDQ2_9BURK | D5X6D8_THIK1 | W0ITR4_9BACT | A0A3A3FU29_9BURK |
| A0A2I2KDM2_9PROT | F8L829_SIMNZ | C4ZLI5_THA5P | Q2LWT2_SYNAS | L9P9K3_9BURK |
| A0A0J6RDC5_9NEIS | A0A2S588Y4_9BURK | A0A1H5UIG0_9RHOO | A0A480AKX6_9BURK | A0A4Q7LFX3_9BURK |
| A0A1Y6BDK2_9PROT | A0A2U8FW21_9BURK | A0A0F3K860_9NEIS | A0A4Q5NY30_9BACT | A0A4R3UH10_PELSC |
| A0A1T4YAJ2_9BACT | K9DHH5_9BURK | A0A257CCL8_9BURK | A0A1G6W3S9_9BURK | A0A4R7S3L8_9BACT |
| A0A1Y0FRK2_9GAMM | A0A2M6VCD0_9BURK | A0A4101JWU7_9BURK | F8L3M9_SIMNZ | A0A2Z6AZ48_9DELT |
| A0A257CRE2_9BURK | A0A4R6QHU7_9BURK | A0A2S8STC3_9BACT | A0A4Q5VDV3_9GAMM | A0A437LLP7_9BURK |
| A0A0Q5Z7E9_9BURK | B6A2I8_RHILW | A0A4Q3LFK3_9BURK | A0A1H3DUR4_9PROT | N6YBF1_9RHOO |
| A0A1D9BIG2_9NEIS | W0S178_9PROT | A0A1Y0G5Y6_9PROT | A0A254TKG9_9BURK | A0A290ZQH8_9RHOO |
| A0A126Z9Q8_9BURK | A0A2M6VQD7_9BURK | A0A0C4YEN6_9BURK | A0A1I1Q7D5_9BURK | A0A142WZ42_9PLAN |
| A0A0P0M7H1_9BURK | D0D610_9RHOB | A0A1I4L0M9_9PROT | A0A431T9N2_9BURK | A0A1H4FC08_9BURK |
| A0A1D9GY60_9BURK | A0A433SE04_9BURK | A0A2Z6I9J0_9BURK | A0A186U7K1_9PROT | A0A0Q6WNM6_9BURK |
| A0A0K6GT67_9NEIS | F3YUZ2_DESAF | A0A239HMA0_9BURK | Q0AF45_NITEC | F8KX54_PARAV |
| A0A1I3ZDK1_9PROT | A0A257DDC0_9BURK | A0A1S8FRH4_9BURK | D0LHN1_HALO1 | F9ZP46_ACICS |
| A6CD16_9PLAN | A0A1G5SDZ5_9PROT | G8QMB4_AZOOP | A0A369CM50_9GAMM | A0A2T6FF09_9GAMM |
| A0A109CQR9_9BACT | A0A1P8WL02_9PLAN | B5YJ50_THEYD | B8FD61_DESAL | A0A1H3K3N2_9PROT |
| A0A2P8KMZ3_9BURK | F2LHD5_BURGS | B9TDF1_RICCO | Q2LSW3_SYNAS | A0A345DDY4_9BURK |
| A0A1H1GHV2_9RHIZ | A0A2P9HA36_PARUW | A0A4R6YBN4_9BURK | A0A098UC71_9BURK | A0A238ZXV8_9DELT |
| A0A0J9E9Q1_9RHOB | A0A4Q1S9M8_9BACT | A0A418X478_9BURK | A0A1H2MW07_9PSED | D6YSC2_WADCW |
| A0A1A8Y0R0_9RHOO | A0A1G8M0B2_9RHOO | A0A0Q9YP50_9COXI | A0A4R3JTX4_9PROT | A0A0F7KK57_9PROT |
| A0A0E3YVM1_9BACT | A0A4R6N3D2_9BURK | A0A328ZU34_9PROT | A4G9P3_HERAR | A0A254NE69_9BURK |
| A0A147GSU4_9BURK | A0A315FGQ6_9BURK | A0A086WDH7_9BURK | A0A194AIB1_9DELT | Q46WJ2_CUPPJ |
| A0A4Q5NX30_9BACT | E5YBJ4_BILWA | D58WA7_PLAL2 | A0A2T4ZVX6_9PROT | A0A1S9AMD8_9BURK |
| A0A0R0MRT3_9BURK | A0A1U9N607_9GAMM | A0A426CHF7_9BACT | A0A4Q1C9W9_9BACT | A0A0K2DHS9_9RHIZ |
| A0A4Q9H4I6_9BURK | A0A418XPX0_9BURK | D3RS21_ALLVD | A0A2T7UB48_9BURK | A0A1D8AXE3_9BACT |
| A0A427E2G4_9PSED | A0A3N1WP35_9BURK | A0A1P8WSD1_9PLAN | A0A1I4NUY5_9BURK | A0A1G6CNW9_9DELT |
| A0A0J7Y0M7_9SPHN | A0A0U5EPV8_9BACT | A0A2R5F6N8_9PROT | A0A1Q2TXJ7_9SYNE | A0A1I2E837_9PROT |
| G9PY68_9BACT | A0A1V1PI45_9DELT | A0A091FG89_9DELT | W0SCQ3_9PROT | F9ZGY4_9PROT |
| A0A1Y2Q430_9SPHN | A0A0A1H9P5_9BURK | A0A1H9NMS2_9PROT | W0SFC8_9PROT | A0A4R6QUU1_9BURK |
| F8GHJ5_NITSI | A0A3M0BZK2_9AQUI | A0A3I8GZD9_9BURK | B5JF49_9BACT | A0A1M5P847_9BURK |
| A0A4P6PHF6_9BACT | A0A368KXE9_9PLAN | C6WSR0_METML | A0A437LLA6_9BURK | A0A080MA98_9PROT |
| A7I273_CAMHC | A0A381DHF6_9PROT | A0A0G3EG67_9BACT | D2QZD1_PIRSD | A0A1H2PVJ6_9BURK |
| A0A1T4VHG9_9DELT | C1DU75_SULAA | A0A0P0MDL0_9BURK | A0A1Z4JM26_LEPBY | A0A4R3UQC1_PELSC |
| A0A1H1SQ64_9BACT | A0A0C5JBE3_9RHOO | A0A1C3JXM2_9BURK | A0A426V8M0_9BURK | A0A254NBE3_9BURK |
| A0A239SWA5_9BURK | D7DHW7_METV0 | A0A117V5E0_9SPHN | L0DMP8_SINAD | T0J4C4_9SPHN |
| A0A1G4TUD8_9SPHN | A0A2V5C7S0_9SPHN | T0HQ23_9SPHN | A0A432MET0_9BACT | A0A0Q68M14_9PROT |
| A0A2N7WZC1_9BURK | D5CNC1_SIDLE | A0A0N1C4F7_9SPHN | A0A0C5JJN9_9RHOO | A0A372JV49_9BURK |
| C6WWI4_METML | M5TVR1_9PLAN | S3XR02_9PROT | M5TB48_9PLAN | A0A0U3F012_9BURK |
| A0A0S3PPK5_9BRAD | A0A117UZY8_9SPUN | A0A257DEJ8_9BURK | A0A3M8R6I4_9PROT | A0A3N7DGN4_9BURK |
| A0A2I2KFV1_9PROT | Q2G9E9_NOVAD | A0A0Q6MEA8_9BURK | A0A1X7M3B3_9BURK | A0A142XV95_9PLAN |
| A0A1R1J544_9RHOO | A0A139BTK5_9PROT | A0A3R8PBZ8_9CAUL | D3DJU4_HYDTT | Q7UX59_RHOBA |
| F4QJ54_9CAUL | A0A1T4WYQ4_9BACT | A0A133XH21_9RHOO | A0A080M934_9PROT | A0A0S4K937_9BURK |
| D9SJ08_GALCS | A0A366H354_9BACT | A0A2T6DZI5_9BACT | A0A1Q8YE91_9BURK | A0A211DIS5_9PROT |
| A0A1A8XDA4_9PROT | B1LX61_METRJ | C6XQ23_HIRB1 | A0A1N6HQH0_9BURK | A0A3D8K4P0_9BURK |
| A0A1X7FJW5_TRICW | A0A437QB99_9GAMM | A0A1H2MU36_9PSED | A0A127F9X3_STEDE | A0A1E2UXH9_9GAMM |
| A0A3R8U3H3_9PSED | A0A1H1UC70_9PSED | A0A4R2KXX8_9GAMM | A0A1G5QCX6_9GAMM | A0A1G9LTH1_9RHOB |
| A0A3B7M7T1_9GAMM | F1VIJ14_9BURK | A0A1Y0FYX5_9GAMM | A0A1I5KFR8_9BURK | A0A4R5W4V7_9BURK |
| D0KWK8_HALNC | A0A0T6W2J7_9ALTE | A0A2I8DK75_9BURK | A0A1D9B990_9NEIS | L2F973_9GAMM |
| A0A0Q5H5L9_9BURK | A0A161HPD4_9BURK | A0A1B7H1Z2_9BURK | A0A437TKY8_9GAMM | A0A1V3SAZ7_9BURK |
| A0A1N6GQD3_9BURK | A0A370N8M0_9BURK | A0A1N6L735_9BURK | C4XH25_DESMP | A0A1H4FWS6_9BURK |
| B2JIP0_PARP8 | A0A0A1H8R9_9BURK | A0A1M5VQM2_9DELT | A0A1G6E9A3_9DELT | A0A1G9M8L8_9FIRM |
| A0A4V2Q8E0_9FIRM | G2IXH9_PSEUL | A0A317MQ69_9GAMM | A0A091FI18_9DELT | O84681_CHLTR |
| A0A4R3MVI1_9GAMM | A0A3M8FT42_9BACT | A0A1B6Y7I1_9BACT | J2U0D9_9BURK | A0A0F5L3W5_9RHIZ |
| A0A1Q2MI71_9BACT | G7UPW0_PSEUP | B0VJ43_CLOAI | A0A1H0DJU8_9DELT | A0A381KS33_CHRVL |
| F1W377_9BURK | A0A254VG95_9BURK | A0A139SNJ9_9BACT | A0A2T5CE71_9DELT | B8DPS5_DESVM |
| A0A2R8BXA8_9RHOB | S3CEZ4_9BURK | A4JSW8_BURVG | Q46XX8_CUPPJ | A0A1I3ICR7_9PLAN |
| A0A1H7P696_9SPHN | MIP6P2_DESSD | A0A2Z5EFG0_9SPHN | A6WWU1_OCHA4 | I6Z3W2_MELRP |
| A0A1I3DSH3_9BURK | A0A2C9CHB6_KUEST | A0A1N6XJ91_9GAMM | A0A077LH04_9PSED | A0A1G9AB34_9PSED |
| A0A1E4UVV0_9PSED | A0A4Q7YKC0_9GAMM | A0A0D1M236_PSEPU | W0HEY3_PSECI | A0A4S3KWN1_9GAMM |
| A0A3A3G9Z0_9BURK | A0A1I4VI28_9GAMM | A0A166XKS3_9GAMM | A0A2P6ARS2_9GAMM | B8GU41_THISH |
| M5ONN9_9GAMM | A0A1H9D5I6_9GAMM | A0A1H6FES3_9GAMM | W6LT80_9GAMM | A0A4R21AB8_9GAMM |
| A0A369CC98_9GAMM | A0A4P9VJO9_9GAMM | A0A363UK93_9GAMM | A0A372DL79_9GAMM | A0A1Q2M9Q1_9GAMM |
| A0A0Q5ZI59_9BURK | A0A0Q7AU18_9BURK | A0A1L6JGT2_9SPHN | A0A1G5DNR9_9GAMM | A0A0G3BPX8_9BURK |
| A0A0K1K0W9_9BURK | A0A1H8IXV3_9BURK | Q221L0_RHOFT | A0A086W5K9_9BURK | A0A191ZHS1_9GAMM |
| A0A1W6L5H9_9BURK | A0A193LFF4_9GAMM | A1VK97_POLNA | A0A2U0SGG9_9SPHN | A0A1Y0N8D3_9BURK |
| A0A1C9VAP8_9BURK | A0A2P8KRI0_9BURK | A0A1Y2R3H9_9BURK | A0A0T2ZC47_9BURK | A0A1G7B8X2_9BURK |
| A0A1H4FCD1_9BURK | A0A3N4UZS4_9BURK | A0A0Q4YM57_9BURK | A0A3M6QJ47_9BURK | A0A418XQD2_9BURK |
| A0A1A8TG45_9GAMM | A0A4R6F1W1_9BURK | A0A147GN38_9BURK | A0A1X7GTF7_TRICW | A0A370UAF6_9GAMM |
| Q13SK8_PARXL | A0A1W1XQV1_9NEIS | A0A2I8DSP4_9BURK | A0A084IQP0_SALHC | A0A3D8K100_9BURK |
| A6GLP8_9BURK | A0A3N6NYZ8_9BURK | F2K4U3_MARMI | A0A1I4NNE9_9BURK | A0A257DAY2_9BURK |
| A0A4R6R6F3_9BURK | A0A060NFK6_9BURK | A0A4Q7LF36_9BURK | A0A3EIRE19_9BURK | A0A0S4M0F7_9BACT |
| A0A2S5SV83_9BURK | A0A257CP66_9BURK | A0A4R6N2I2_9BURK | A0A3A5J188_9GAMM | S7V316_DESML |
| A0A1I6KF98_9BURK | B8FE34_DESAL | I2K5N5_9PROT | E5Y4E7_BILWA | A0A1L3GPN4_9DELT |
| A0A127ETV6_9RHIZ | T2GGI0_DESGG | A0A177PJJ3_9RHIZ | A0A1T4WJG8_9DELT | A0A177P8L6_9RHIZ |
| A0A0S3QVR1_THET7 | A0A1I2IE98_9DELT | A0A0E2JA97_CHLPS | I0IQ01_LEPFC | A0A225DWB5_9BACT |
| A0A1L6M3N5_9DELT | A0A2M8ZS71_9BRAD | A0A4Q8QXT9_9BRAD | Q1QQ72_NITHX | Q89HG9_BRADU |
| A0A1B7XB79_9DELT | A0A1L4CZB1_9PROT | A0A0D1P1I4_BRAEL | A0A2T5C7Z4_9DELT | A0A3P1XFL5_9BURK |
| A6FZI7_9DELT | B1Y075_LEPCP | C6HW33_9BACT | A0A1H2EHR9_9BACT | A6T3F7_JANMA |
| A0A1V4I184_NITVU | H5WL41_9BURK | A0A0Q8R6U0_9SPHN | A0A3M2I1E1_9GAMM | I1AQX3_9RHOB |
| A0A1I4XR60_9NEIS | A0A4Q2IYD0_9BURK | A0A0Q8RHK1_9BURK | Q0FN98_SALBH | A0A2K9LNQ5_9GAMM |
| Q72CL0_DESVH | A0A1H2XJ02_9RHOB | A0A0Q9YK08_9COXI | D8PF27_9BACT | A0A4R5MFW6_9BURK |
| A0A257FUH6_9BURK | A0A4R6R6X1_9BURK | A0A41IPG51_9GAMM | A0A2U3QGE9_9BACT | A0A1H8CRD3_9RHOB |
| A0A0B6X0X9_9BACT | A0A1T4PVA2_9RHIZ | A0A2Z4Y1C8_9GAMM | A0A286RDF2_9PLAN | A0A1L6JEV0_9SPHN |
| B4D845_9BACT | A0A397NJ75_9SPHN | A0A126P0P9_9BRAD | A0A2T6F3D6_9BURK | A5PCF3_9SPHN |
| A0A059ITW0_9RHOB | A0A2J6MUT4_9BURK | A0A103E9K5_9BURK | A0A363RU93_9BURK | A0A418Q3F8_9SPHN |
| A0A432V196_9SPHN | A0A2V3VL55_9PROT | A0A0Q4X7A7_9RHIZ | A0A345YDF9_9SPHN | A0A0U1Q2L6_9BURK |
| A0A369TIG4_9RHOB | Q5P398_AROAE | A0A2A4G2V4_9SPHN | A0A1W7M8X0_9SPHN | A0A1I6JU85_9SPHN |
| A0A0K1JBD2_9RHOO | A0A1L3JCZS_9SPHN | A0A1I5UED0_9SPHN | H0PVW3_9RHOO | A0A0A0EGN8_9RHOB |
| A0A0Q4C8I1_9SPHN | Q02A68_SOLUE | A0A0N1ANI3_9SPHN | A0A0Q5QRQ2_9SPHN | A0A1Y0EL89_9BURK |
| A0A369VYL3_9SPHN | A0A1W1X6E3_9DELT | N9W1M4_9SPHN | A0A127EZQ7_9RHIZ | F8JBG0_HYPSM |
| A0A0T2QHZ3_9SPHN | A0A3F2U9S6_9RHIZ | A0A4R5LY34_9BURK | A0A432MA36_9GAMM | A0A437M1R8_9PROT |
| A0A1J7MVF4_9RHIZ | A0A1I6L623_9BURK | A0A1U7CUQ1_9BACT | G6E8H9_9SPHN | V4YJF7_9PROT |
| D1Y3MS_9BACT | A0A143B6N9_9DELT | A0A147EEZ7_9SPHN | A0A1W1ZKZ7_9SPHN | B8IDY5_METNO |
| A0A495BLJ2_9BACT | A0A0T0PSA5_9SPHN | A0A1E3LSX6_9SPHN | A0A1M6DL45_9FLAO | A9BV48_DELAS |
| V5SD10_9RHIZ | A0A1L6FCD4_9RHOO | A0A2S4LY23_9BURK | A0A2A4B4E7_9SPHN | A0A0P0A7B5_9RHOB |
| A0A4R1L5R2_9BACT | A3WAB6_9SPHN | A0A069PKP0_9BURK | C5A9K2_BURGB | A0A1U7CZ16_9BACT |
| A0A0D1MIU7_BRAEL | A0A2T6DBV4_9BACT | A0A2N7VYF8_9BURK | A4IPT7_BURVG | A0A370MYG1_9BURK |
| U5QIR9_9CYAN | A0A1I6JF19_9ALTE | A0A1E3M056_9SPHN | A0A0M4D0G8_9DELT | F2NGR3_DESAR |
| A0A1G5B504_9GAMM | Q2G866_NOVAD | A0A2T5RAA3_9DELT | A0A2Z5EHS4_9SPHN | A0A494TKX8_9SPHN |
| A0A0B1ZU05_9SPHN | A0A4V1AD88_9SPHN | A0A345WLY2_9SPHN | A0A1G5SSK7_9SPHN | A0A4R6FRF0_9SPHN |
| G8QN05_AZOOF | Q0VSG7_ALCBS | A0A1Z4VPQ3_9GAMM | A0A2H6AK05_9BACT | Q1N1M8_9GAMM |
| Q08NT3_STIAD | A0A4R3N006_9GAMM | A0A0H4KU33_9RHOB | A0A2A4B688_9SPHN | A0A1N7NAK4_9RHOB |
| T0SKZ6_9PROT | A0A371WF82_9RHIZ | A0A1H9H411_9GAMM | A0A061SS22_9RHOB | A5VFY9_SPHWW |
| A0A0B4EBY6_9RHOB | A0A2N0H6E3_9SPHN | A0A2R8AY47_9RHOB | B5EGL2_GEOBB | X6L3C0_9RHOB |
| A0A254QR79_9RHOB | A0A0M9EHC5_9RHOB | A0A0P1H3K3_9RHOB | B5EI39_GEOBB | A3JR75_9RHOB |
| A0A1X6YCF3_9RHOB | A0A238JA78_9RHOB | A0A1X4NML0_9RHOB | T0I8E9_9SPHN | A0A0C1JMD5_9BACT |
| B8ERR9_METSB | A0A0F4RFD2_9RHOB | A0A1P8WHB3_9PLAN | A0A318T3Z7_9RHOB | A0A2T5R7V2_9DELT |
| A0A368KZ82_9BURK | A0A222FE02_9RHOB | A0A1T5JTU9_9GAMM | A0A1I6UF39_9RHOB | A0A411ZA14_9SPHN |
| A0A0F5P9W7_9SPHN | A0A1Y5SVV3_9RHOB | A0A246QVN4_9RHOB | B6B180_9RHOB | I3ZET2_TERRK |
| A0A2A2AFN7_9BURK | A0A0J9E9H3_9RHOB | A0A1G6LRX6_9SPHN | G6Y699_9RHIZ | B5EAZ0_GEOBB |
| A0A1G5DMA0_9GAMM | A0A4Q6C0R0_9GAMM | D5AMX8_RHOCB | A0A2N3LU09_9RHIZ | A0A1T2LBG8_9GAMM |
| E8R5C8_ISOPI | A0A2Z6ESQ2_9BURK | A0A0K0Y2W7_9RHOB | A0A432DQK3_9BURK | A0A0G9IBV0_9XANT |
| A0A1H8UVI0_9RHOB | A0A1H1C0Y6_9GAMM | A0A0A8WUF5_9DELT | A0A396RPY1_9SPHN | A0A2V4MMV1_9RHOB |
| A0A0H4L4Q1_9RHOB | A0A0K1JBG6_9RHOO | D8JQZ2_HYPDA | A0A225NHG2_9RHOB | A0A2R7IJ32_9SPHN |
| A0A165RQ90_9SPHN | A9E149_9RHOB | C9CZK8_9RHOB | X7FFY0_9RHOB | A0A147IKQ2_9SPHN |
| A0A346A173_9RHIZ | A0A328B460_9CAUL | J3C9I4_9BURK | A0A225E4G8_9BACT | J5PQF1_9RHOB |
| A0A1H2R6H7_9RHOB | Q5H3Q6_XANOR | A0A1Y5S5T4_9RHOB | A0A1J4WXN6_9NEIS | A0A0Q4FFX2_9SPHN |
| G8NQH3_GRAMM | B4R9E7_PHEZH | A0A430BJX8_9SPHN | A0A0S4K8G5_9BURK | A0A1V4I2Z1_NITVU |
| A0A4Q1JUX1_9GAMM | I7ZBF3_9GAMM | A0A49BDPE4_9PROT | B3E9T4_GEOLS | A0A0E9MQ78_9SPHN |
| C4XKD6_DESMR | A0A1M6H1D4_9BACT | A0A0Q8XKF5_9SPHN | A0A0P6ZKY5_9SPHN | G7QARS_9DELT |
| A0A2K1ECN7_9SPHN | A0A418PZ09_9SPHN | A0A2T6BLG4_9RHOB | E3I3D9_RHOVT | A0A494X5V6_9BURK |
| A1AMQ3_PELPD | A0A3M6QTJ8_9BURK | A0A3F2V589_9GAMM | A0A418NHY2_9SPHN | Q5H4L0_XANOR |
| Q74C96_GEOSL | A0A0M4MH57_9SPHN | A0A4R0NZJ9_9SPHN | E7RYV3_9BURK | A0A0H4VFC8_9SPHN |
| A0A410UQI1_9BURK | A6Q3Q7_NITSB | A0A0N1BES6_9SPHN | A0A0N0LWL8_9SPHN | L0GUK8_9GAMM |
| A0A494XLJ1_9BURK | F3KS43_9BURK | A0A2U2J4K8_9SPHN | G7Q8W6_9DELT | A0A3A9JHW7_9PROT |
| A0A2T6CNH0_9BACT | C4XJ01_DESMR | A0A3M0CXX9_9PROT | Y0KEC5_9PROT | A0A0D6JET8_9RHIZ |
| T2G7Z5_DESGG | N6X5G0_9RHOO | A0A059FQ02_9RHOB | A0A317F8B8_9PROT | A0A1X7FYY6_9SPHN |
| Q2ND45_ERYLH | A0A2S7TX63_9BACT | A0A2N4WVZ8_9SPHN | A0A1C7DAS6_9SPHN | A0A437H186_9SPHN |
| V4PZC5_9CAUL | Y0KFR6_9PROT | A0A126R8Q9_9SPHN | A0A1Z1F9A8_9SPHN | A0A0G3X5J9_9SPHN |
| A0A2L0AF85_9SPHN | A0A4R7DAF2_9SPHN | A0A0Q7UIX8_9CAUL | A0A0Q4LR07_9SPHN | V4TF68_9RHIZ |
| A0A250KL85_9GAMM | A0A0W1QHA8_9SPHN | A0A220W203_9SPHN | F2ND55_DESAR | A0A125QIC4_9SPHN |
| A0A2N0HKM8_9SPHN | A0A062TUS7_9RHOB | A0A0S9CK44_9SPHN | A0A2N7VUC9_9BURK | A0A1H9HXT8_9GAMM |
| A0A3S2VBS8_9SPHN | A0A1H7YWK3_9SPHN | A0A1D8AUR4_9BACT | A0A257J994_9PROT | A0A1Q8I5B4_9BURK |
| A0A1B2AGP5_9SPHN | A0A0X8HGQ8_9GAMM | T0H520_9SPHN | A0A1I1HRD9_9BURK | A0A2K2FVX5_9SPHN |
| A0A4R3TI86_9SPHN | A0A0D6B191_RHOSU | A0A399RL44_9RHOB | A0A2N7TPR3_9GAMM | A0A2A8I0M9_9SPHN |
| A0A1W6LPZ7_9BACT | A0A3S0HQH0_9GAMM | A0A0B8ZCP3_9SPHN | A0A437J9X6_9SPHN | A0A2A4I9I0_9SPHN |
| A0A369Q7Y8_9SPHN | A0A1B6ZBV1_9SPHN | A0A419RTD4_9SPHN | A0A0A8K2H7_9RHIZ | N6Y4G4_9RHOO |
| A0A192D3D5_9SPHN | A0A2N7X9Z5_9BURK | A0A418NVA4_9SPHN | G0JRZ5_9PROT | A0A1Y6EHK5_9SPHN |
| A0A3D8JS29_9BURK | A0A2S8B5F6_9SPHN | A0A1I2ZMM4_9GAMM | A3UFK0_9RHOB | S2L420_HALAF |
| E5APR7_PARRH | N6YZQ4_9RHOO | T0IQM7_9SPHN | A0A1Z4RKC7_9CYAN | A0A1N6GWJ3_9SPHN |
| A0A0X8WSW1_9CYAN | A0A0S3F481_9SPHN | F2NFB2_DESAR | B1ZTI4_OPITP | E8WXZ7_GRATM |
| A0A157SWJ7_9BORD | A0A0M4D1X9_SPHS1 | A0A1G5QG76_9GAMM | L0DMH3_SINAD | A0A2Z4UQ62_9RHIZ |
| H7C7I8_CAUVC | A0A371X8G2_9RHIZ | A0A251WFZ7_9CYAN | B0SYF0_CAUSK | Q1IRM5_KORVE |
| A0A3R9EEY9_9VIBR | G4RFS3_PELHB | A0A0Q5VM15_9CAUL | A0A257CBR2_9BURK | K9UQ51_CHAP6 |
| A0A1H6AXF7_9BACT | A0A1M5DH29_9BACT | K9WKN1_9CYAN | A0A2A2G9L3_9BACT | K7VZW7_9NOST |
| A0A1V1V1P8_9CAUL | A0A0C1RAH3_9CYAN | A0A178IR14_9BACT | Q7NEJ4_GLOVI | A0A2R5F8T2_9PROT |
| A0A2T6B917_9RHOB | A0A1M4URY6_9BURK | A0A0J5Q533_9RHOB | A0A3T0N9F1_9RHOB | A0A2T5ME23_9GAMM |
| H0HXF0_9RHIZ | A0A254QVH9_9RHOB | A0A090FD34_9RHIZ | I4YX58_9RHIZ | A0A1Y5RIT1_9RHOB |
| A0A1N7FJ07_9RHOB | A0A3N2QUT2_9RHOB | A0A1H9MJS6_9GAMM | A0A0S4KN73_9BACT | Q1LH50_CUPMC |
| A0A0C1YVU8_9BURK | A0A420DPH3_9RHOB | Q2CIE5_OCEGH | A0A1Y5T2I1_9RHOB | A0A255RAT4_9PLAN |
| A0A1I3V8D5_9BURK | A0A1G6W062_9BURK | A0A4Q3YE26_9RHOB | M5RQL8_9PLAN | A0A1Y1SHS6_9GAMM |
| A0A1I6MRC4_9BACT | A0A286RKY0_9PLAN | Q46Q30_CUPPJ | A0A0U3VLR0_9BURK | A0A1D9HBI2_9BURK |
| A3TZ38_PSEBH | A0A0F5VBV7_9GAMM | A0A077LFR9_9PSED | A0A1G4SNS1_9CAUL | A0A1G7PBE2_9BACT |
| A0A0QJERH6_9RHOB | A0A0T1WSA0_9RHIZ | A0A1H7ANJ7_9RHOB | A0A2R8AIL7_9RHOB | A0A0D1PLC5_PSEPU |
| A0A395LSK3_9SPHN | A0A01IMID5_9PROT | A0A370N0T5_9BURK | A0A0R0DYY3_9GAMM | A0A1N6HAH0_9BURK |
| A0A1X7F531_TRICW | A0A1N7SBT4_9BURK | A0LID0_SYNFM | A0A1I6JGG6_9ALTE | A0A3M8FQ32_9BACT |
| A0A1N6EAT4_9RHOB | A0A1I7FIY1_9GAMM | A0A2R7LBE4_9CAUL | A4SYN1_POLAQ | . |

The present disclosure further provides a protein nanopore, wherein the protein nanopore contains cap gate and central gate structures; and
an amino acid sequence of the protein nanopore is any one of amino acid sequences screened by the screening method.

In some embodiments, the protein nanopore is a polymer composed of monomeric proteins of any one of the amino acid sequences.

In some embodiments, the polymer includes 1216-mers (dodecamer-hexadecamer).

In some embodiments, the protein nanopore contains a central gate signature sequence, a cap gate signature sequence, and an isoelectric point determination sequence.

In some embodiments, the protein nanopore contains a central gate signature sequence, or a cap gate signature sequence, or an isoelectric point determination sequence.

In some embodiments, the isoelectric point determination sequence is an amino acid sequence represented by SEQ ID NO.5 or a sequence having homology greater than 75% to SEQ ID NO.5, wherein
the SEQ ID NO.5 sequence is:

KAKITVGEDVPFITGQSQTVGGNVMTMIQRQNVGIT;

In some embodiments, the cap gate signature sequence is an amino acid sequence represented by SEQ ID NO.6, SEQ ID NO.10, SEQ ID NO.11 or SEQ ID NO.12 or a sequence having homology greater than 75% to SEQ ID NO.6, SEQ ID NO.10, SEQ ID NO.11 or SEQ ID NO.12, wherein the SEQ ID NO.6 sequence is:

GATGASSLSGSTTGAAGSLGVVSGAAGAASALSG,

the SEQ ID NO.10 sequence is:

RTRKEPDDITYRTDAAGQPIYNNNGNRVIASITEGKEIQGDFG,

the SEQ ID NO.11 sequence is:

GPRNVATVPLGQDLTQPPVAGTG,

and
the SEQ ID NO.12 sequence is:

GNIVVDANGNAVTQTTSTQGDFTALASLLGGLNG.

In some embodiments, the central gate signature sequence is an amino acid sequence represented by SEQ ID NO.7, SEQ ID NO.8, SEQ ID NO.13, SEQ ID NO.14 or SEQ ID NO.15, or a sequence having homology greater than 75% to SEQ ID NO.7, SEQ ID NO.8, SEQ ID NO.13, SEQ ID NO.14 or SEQ ID NO.15, wherein the SEQ ID NO.7 sequence is:

QSQTVGGNVMTMIQ;

the SEQ ID NO.8 sequence is:

QTITALTNASQLIGTMAVGPTTT,

the SEQ ID NO.13 sequence is:

PTITGATASTNNTNPFQTVERK,

the SEQ ID NO.14 sequence is:

QVPILQALAAGNAAFQNVTY,

and
the SEQ ID NO.15 sequence is:

PILTGTTASAGSSNPATTVDRQ.

In some embodiments, the protein nanopore contains a modification structure.

In some embodiments, positions modified by the modification structure include a central gate, a cap gate, N-terminal, or C-terminal.

In some embodiments, modification of the modification structure includes at least one of the following: 1) adding at least one amino acid or unnatural amino acid, 2) reducing at least one amino acid; and 3) performing substituting or modifying on a side chain on at least one amino acid in the modification structure.

The present disclosure further provides a single-pore protein nanopore, wherein the single-pore protein nanopore is obtained in a following manner: making one or more deletions to 5262-G322 segment of the protein nanopore according to any one of the above, and removing a cap gate region.

The present disclosure provides nucleotide sequence, wherein the nucleotide sequence encodes the amino acid sequence screened by the screening method, or the nucleotide sequence encodes the protein nanopore according to any one of the above.

The present disclosure further provides a recombinant vector, an expression cassette or a recombinant bacterium containing the nucleotide sequence.

The present disclosure further provides application of the screening method, the protein nanopore, the nucleotide sequence or the recombinant vector, the expression cassette or the recombinant bacterium according to any one of the above in detecting an electrical and/or optical signal of an object to be detected.

The present disclosure further provides application of the single-pore protein nanopore in detecting an electrical and/or optical signal of an object to be detected.

In some embodiments, the application includes following steps:
preparing a biochip containing a protein nanopore by embedding the protein nanopore in a phospholipid bilayer, and by means of a computer processor and a sensing device, after adding an object to be detected, recording electrical signals and/or optical signals at two ends of the biochip, wherein
the object to be detected includes any one or a combination of at least two of a nucleic acid, a protein, a polysaccharide, a neurotransmitter, a chiral compound, a heavy metal, and a toxin.

The present disclosure further provides a method for detecting an electrical and/or optical signal of an object to be detected, wherein the method includes:
obtaining a final sequence of an animo acid sequence of a protein nanopore obtained according to the screening method, preparing a biochip containing the protein nanopore using the protein nanopore having the final sequence by embedding the protein nanopore into a phospholipid bilayer, and by means of a computer processor and a sensing device, after adding an object to be detected, recording electrical signals and/or optical signals at two ends of the biochip, wherein
the object to be detected includes any one or a combination of at least two of a nucleic acid, a protein, a polysaccharide, a neurotransmitter, a chiral compound, a heavy metal, and a toxin.

The present disclosure further provides a device for screening an amino acid sequence of a protein nanopore, wherein the device includes:
an evaluation module, configured to acquire amino acid information about a known protein nanopore, and evaluate a signature sequence of a dual-pore structure by means of a multiple sequence alignment algorithm;
a data processing module, configured to use a hidden Markov model to search for amino acid sequence information matched with the signature sequence of the dual-pore structure, and remove redundant data information;
a locating and screening module, configured to locate and screen amino acid sequences obtained from the data processing module to obtain candidate sequences;
a calculation module, configured to calculate a matching length and an envelope length of the candidate sequences; and
a registration analysis module, configured to perform registration on the candidate sequences by means of the multiple sequence alignment algorithm, calculate a relative mismatch relationship with the known protein nanopore, and analyze a structure of the candidate sequences to obtain a final sequence.

The present disclosure provides a system for screening an amino acid sequence of a protein nanopore, including:
one or more processors; and
a storage device, configured to store one or more programs, wherein
when the one or more programs are executed by the one or more processors, the one or more processors implement the screening method for an amino acid sequence of a protein nanopore.

The present disclosure further provides a computer storage medium, on which a computer program is stored, wherein the computer program implements, when being executed by a processor, the screening method for an amino acid sequence of a protein nanopore.

### BRIEF DESCRIPTION OF DRAWINGS

FIG. 1 is a schematic diagram of lengths of an initial candidate sequence obtained by taking VcGspD (PDB: 5WQ8) as a template sequence in Example 1, which from top to bottom are a matching length of a template sequence (QUERY) matching part, a length of a candidate sequence (TARGET) matching part, and an envelope length (TARGET ENVELOPE) of the candidate sequence, respectively.

FIG. 2 is a schematic diagram of a mismatch relationship between the candidate sequence and VcGspD after MAFFT alignment in Example 1, in which two upper dotted lines are mismatch values (−4˜0) of four known dual-pore structures, and remaining lower dotted lines are mismatch values of known single-pore secretory channels.

FIG. 3 is a graph of a radius relationship between a sequence screened in Example 1 and a VcGspD channel, in which VcGspD-PDB represents a channel size of VcGspD in PDB, VcGspD-Predicted represents a size of VcGspD after calculation and analysis, and LfGspD-Predicted represents a size of LfGspD after calculation and analysis.

FIG. 4 is a structure prediction diagram of a protein nanopore provided in the present disclosure.

FIG. 5 is a structure prediction diagram of a protein nanopore formed from protein VcGspD in V. cholerae.

FIG. 6 is a schematic diagram of DNA translocation through a mutant protein nanopore.

FIG. 7 is a schematic diagram of DNA translocation through wild-type porin.

FIG. 8 provides a structure analysis diagram of monomeric proteins formed from protein sequence C6HW33_9 BACT provided in the present disclosure.

FIG. 9 is a structure analysis diagram of protein VcGspD in V. cholerae.

FIG. 10 is a schematic diagram of a channel width obtained after analyzing 15-mer (pentadecamer) provided in the present disclosure with SWISS-model.

FIG. 11 is a schematic diagram of a channel width obtained after analyzing protein VcGspD 15-mer with SWISS-model.

FIG. 12 shows pore diameter analysis graphs of protein nanopores of VcGspD, ETEC_GspD, and InvG.

FIG. 13 is a purified protein nanopore C6HW33_9 BACT silver stain diagram (L: bacterial lysate; p: Ni-NTA purified protein; 10, 11, 12: polymeric protein isolated by molecular sieve.

FIG. 14 is an electrophysiological diagram of protein nanopore C6HW33_9 BACT.

FIG. 15 shows electrophysiological statistical and IV graphs of protein nanopore C6HW33_9BACT.

FIG. 16 are four protein monomer structures of U3AQV9_9 VIBR (Vibrio), A0A0J8GPG7_9 ALTE (Cate), C7R8G0_KANKD (Kang) and A0A0E9MQ78_9 SPHN (Sphi) predicted based on AlphaFold v2 and Hermite.

FIG. 17 is an immunoblotting assay diagram of proteins obtained from purification of Vibrio, Cate, Kang, and Sphi.

FIG. 18 shows electrophysiological statistical and IV diagrams of Cate channel protein.

FIG. 19 shows electrophysiological statistical and IV diagrams of Sphi protein.

FIG. 20 shows electrophysiological statistical and IV diagrams of Kang protein.

FIG. 21 shows electrophysiological statistical and IV diagrams of Vibrio protein.

### DETAILED DESCRIPTION OF EMBODIMENTS

Technical solutions of the present disclosure are further described below through embodiments and examples in combination with drawings. However, the following embodiments and examples are merely simple instances of the present disclosure, and do not represent or limit the scope of protection of the present disclosure, and the scope of protection of the present disclosure is determined by the claims.

An embodiment of the present disclosure provides a screening method for an amino acid sequence of a protein nanopore. The screening method includes the following steps:
(1) acquiring amino acid information about a known protein nanopore, and obtaining a signature sequence of a dual-pore structure by means of a multiple sequence alignment algorithm;
(2) using a hidden Markov model to search for amino acid sequence information matched with the signature sequence of the dual-pore structure, and removing redundant data information;
(3) locating and screening amino acid sequences obtained in step (2) to obtain candidate sequences, and calculating a matching length and an envelope length of the candidate sequences; and
(4) performing registration on the candidate sequences by means of the multiple sequence alignment algorithm, calculating a relative mismatch relationship with the known protein nanopore, and analyzing a structure of the candidate sequences to obtain a final sequence.

According to the screening method provided in the present disclosure, firstly, the amino acid information about known domain sequences of T2SS and T3SS is searched and obtained from a database. The signature sequence of the dual-pore structure is obtained from these amino acid sequences by means of the multiple sequence alignment algorithm. The amino acid sequence information matched with the dual-pore structure template is searched by the hidden Markov model HMMER v3.3 or HmmerWeb v2.41.1. Then conserved matching regions of the candidate sequences are located and screened by scripts, to obtain the candidate sequences, and the matching length and the envelope length of the candidate sequences are calculated. All the candidate sequences are registered by means of the multiple sequence alignment algorithm (MAFFT v7.273), and a relative mismatch relationship with the known protein nanopore can be calculated. At the same time, all candidate sequences are subjected to structural analysis by taking the sequence of secretin domain of the known protein nanopore as a template with MODELLER v10.1 and HOLE2 v2.2.005. The final sequence obtained by the above screening method has a highly controllable central gate narrow channel and a cap gate channel, and can be used as a novel protein nanopore.

As an optional embodiment of the present disclosure, the signature sequence of the dual-pore structure in step (1) is any one of the amino acid sequences represented by protein SEQ ID NO.1˜4.

SEQ ID NO.1-4 (in which underlined bold parts are sequences of cap gate and central gate regions, and italic bold parts are framework structure conserved regions) are shown as follows:

SEQ ID NO. 1 (PDB: 5WQ8):
KDTTQTKAVYDTNNNFLRNETTTTKGDYTKLAS--ALSSIQGAAVSIAMGD-
WTALINAVSNDSSSNILSSPSITVMDNGEASFIVGEEVPVITGS--TAGSNNDNPFQ;
SEQ ID NO. 2 (PDB: 6I1Y):
KDKTVTDSRWNSDTDKYEPYSRTEAGDYSTLAA--ALAGVNGAAMSLVMGD-
WTALISAVSSDSNSNILSSPSITVMDNGEASFIVGEEVPVITGS--TAGSNNDNPFQ;
SEQ ID NO. 3 (PDB: 5W68):
KPQKGSTVISENGATTINPDTN---GDLSTLAQ--LLSGFSGTAVGVVKGD-
WMALVQAVKNDSSSNVLSTPSITTLDNQEAFFMVGQDVPVLTGS--TVGSNNSNPFN;
SEQ ID NO. 4 (PDB: 5ZDH):
KPQKGSTVISENGATTINPDTN---GDLSTLAQ--LLSGFSGTAVGVVKGD-
WMALVQAVKNDSSSNVLSTPSITTLDNQEAFFMVGQDVPVLTGS--TVGSNNSNPFN.

Optionally, the conserved match regions used for locating and screening the candidate sequences in step (3) are KDT and LAS.

Optionally, the final sequence in step (4) has similarity of 75% or less to the known protein nanopores, for example, the similarity may be 30%-75%, 35%-70% or 40%-60%, such as 75%, 70%, 65%, 60%, 55%, 50%, 45%, 40% or 35%.

Optionally, the amino acid sequences screened by the above screening method are as shown in Table 1:

The amino acid sequences provided in the present disclosure are derived from microorganisms in extreme environments, and have the similarity of less than 75% and even less than 50% to complete sequences and core sequences of known type II (T2SS) and type III (T3SS) secretin proteins. The amino acid sequences can form the protein nanopore structure, and the protein nanopore obtained has an inner wall and an outer wall, wherein the outer wall thereof forms a columnar pore structure, and the inner wall forms a defined dual-pore structure, which is a new system having two reading units.

An embodiment of the present disclosure further provides a protein nanopore. The protein nanopore contains cap gate and central gate structures, and an amino acid sequence thereof is any one of the amino acid sequences screened by the above screening method.

Compared with the nanopore formed from a protein VcGspD, a nanopore formed from an amino acid sequence having more than 95% homology to the VcGspD, a complex CsgG-CsgF, and the like, the amino acid sequence specific to the protein nanopore provided in the present disclosure reduces an inner diameter of the pore, so that a pore diameter of a channel thereof is relatively small.

According to a predicted protein structure, it can be seen that the protein nanopore provided in the present disclosure is newly added with a small segment of helical structure in the cap gate region, and a longer junction fragment in the central gate region. In addition, compared with interaction between N3 terminal and S region via a hydrogen bond in VcGspD, the monomeric protein of the protein nanopore provided in the present disclosure is simpler at the N3 terminal. Besides, the sequence specific to the protein nanopore also changes charges around the pore, has a higher isoelectric point, enhances selectivity of the pore, and significantly reduces an error rate when detecting long repetitive base sequences.

As an optional embodiment of the present disclosure, the protein nanopore is a polymer composed of monomeric proteins of any one of the amino acid sequences.

Optionally, the polymer includes 1216-mers. An oligomer (for example, 12-mer, 14-mer, 15-mer, or 16-mer) that can be assembled from monomeric proteins expressed by the amino acid sequences provided in the present disclosure forms a nanopore channel, and has less than 50% similarity to reported protein nanopore sequences. This protein can be used to prepare nanopore channels.

Compared with reported GspD and InvG, an assembling process of the protein obtained by the screening in the present disclosure is simpler, simplifying the complexity of forming the nanopore channel.

In some embodiments, an isoelectric point of the protein nanopore provided in the present disclosure is 9.71. The protein nanopore in the present disclosure can perform substance detection within a larger pH range than GspD and InvG (isoelectric point smaller than 7).

In some embodiments, the oligomer is 1216-mers. In some embodiments, the oligomer assembled from the monomeric proteins expressed by the amino acid sequences of the present disclosure is generally 12-mer, 14-mer, 15-mer or 16-mer.

Optionally, the protein nanopore contains a central gate signature sequence, a cap gate signature sequence, and an isoelectric point determination sequence.

In some embodiments, the protein nanopore in the present disclosure has more perfect cap gate and central gate amino acid sequences, and can further improve precision of gating regions and improve accuracy of detection. Meanwhile, this also provides a wider range of amino acid site selection for transformation of the protein nanopore.

Optionally, the isoelectric point determination sequence is an amino acid sequence represented by SEQ ID NO.5 or a sequence having homology greater than 75% to SEQ ID NO.5.

In the above, the SEQ ID NO.5 sequence is:

KAKITVGEDVPFITGQSQTVGGNVMTMIQRQNVGIT.

For example, the amino acid sequence of the isoelectric point determination sequence has homology greater than 75%, 77%, 80%, 82%, 85%, 87%, 90%, 93%, 95%, 97%, 99% to SEQ ID NO.5.

Optionally, the cap gate signature sequence is an amino acid sequence represented by SEQ ID NO.6 or a sequence having homology greater than 75% to SEQ ID NO.6.

In the above, the SEQ ID NO.6 sequence is:

5′-GATGASSLSGSTTGAAGSLGVVSGAAGAASALSG-3′.

For example, the amino acid sequence of the cap gate signature sequence has homology greater than 75%, 77%, 80%, 82%, 85%, 87%, 90%, 93%, 95%, 97%, 99% to SEQ ID NO.6.

Optionally, the central gate signature sequence is an amino acid sequence represented by SEQ ID NO.7 or SEQ ID NO.8, or a sequence having homology greater than 75% to SEQ ID NO.7 or SEQ ID NO.8.

In the above, the SEQ ID NO.7 sequence is:

QSQTVGGNVMTMIQ.

In the above, the SEQ ID NO.8 sequence is:

QTITALTNASQLIGTMAVGPTTT.

For example, the amino acid sequence of the central gate signature sequence has homology greater than 75%, 77%, 80%, 82%, 85%, 87%, 90%, 93%, 95%, 97%, 99% to SEQ ID NO.7 or SEQ ID NO.8.

In addition, in the present disclosure, the protein nanopore further contains a modification structure. The sequence structure reduces the inner diameter of the pore, changes the charges around the pore, and enhances the selectivity of the pore. In addition, the pore region is neutral amino acid without charges.

Optionally, positions modified by the modification structure include the central gate, the cap gate, N-terminal, or C-terminal.

Optionally, modification of the modification structure includes at least one of the following: 1) adding at least one amino acid or unnatural amino acid, 2) reducing at least one amino acid; and 3) performing substituting or modifying on a side chain on at least one amino acid in the modification structure.

As an example, in some embodiments, amino acids 274 and 279 on a cavity of the protein nanopore are G, specifically forming a α-helical structure on a cavity wall.

In some embodiments, the pore can be changed into a single-pore protein nanopore by making one or more deletions to S262-G322 segment, and removing the cap gate region. In some embodiments, it is also possible to make insertion into the sequence or mutate one or more amino acids of the sequence, to change a size and stability of a cap gate pore.

In some embodiments, insertion, mutation, and deletion are made in V416-T447, to change a size of a central pore. In some embodiments, adjustment of the central pore can also be achieved through insertion, mutation, and deletion to K364-T403.

An embodiment of the present disclosure provides a nucleotide sequence, the nucleotide sequence encoding the amino acid sequence screened by the above screening method, or the nucleotide sequence encoding the above protein nanopore.

An embodiment of the present disclosure further provides a recombinant vector, an expression cassette or a recombinant bacterium including the above nucleotide sequence.

An embodiment of the present disclosure further provides application of the above protein nanopore, the above recombinant vector, the expression cassette or the recombinant bacterium in detecting an electrical signal of an object to be detected.

An embodiment of the present disclosure further provides application of the above single-pore protein nanopore in detecting an electrical and/or optical signal of an object to be detected.

Optionally, the above application further includes following steps:
preparing a biochip containing a protein nanopore, formed by embedding the protein nanopore in a phospholipid bilayer, and by means of a computer processor and a sensing device, after adding an object to be detected, recording electrical signals at two ends of the biochip.

In the above, the object to be detected includes any one or a combination of at least two of a nucleic acid, a protein, a polysaccharide, a neurotransmitter, a chiral compound, a heavy metal, and a toxin.

The present disclosure also provides an example method for using a protein nanopore. The method includes: preparing a biochip, formed by embedding the protein nanopore into a phospholipid bilayer and an analogue thereof; by means of a computer processor and a sensing device, after adding an object to be detected, recording electrical signals at two ends of the chip, and reflecting information about the object to be detected by the electrical signals. Optionally, a sample for substance detection includes any one of a nucleic acid, a protein, a polysaccharide, a neurotransmitter, a chiral compound, a heavy metal, and a toxin and combinations thereof.

An embodiment of the present disclosure provides a method for detecting an electrical and/or optical signal of an object to be detected. The method includes:
obtaining a final sequence of an amino acid sequence of a protein nanopore by the above screening method, preparing a biochip containing the protein nanopore using the protein nanopore having the final sequence, by embedding the protein nanopore in the phospholipid bilayer, and by means of a computer processor and a sensing device, after adding an object to be detected, recording electrical signals and/or optical signals at two ends of the biochip.

In the above, the object to be detected includes any one or a combination of at least two of a nucleic acid, a protein, a polysaccharide, a neurotransmitter, a chiral compound, a heavy metal, and a toxin.

An embodiment of the present disclosure provides a device for screening an amino acid sequence of a protein nanopore. The device includes:
an evaluation module, configured to acquire amino acid information about a known protein nanopore, and evaluate a signature sequence of a dual-pore structure by means of a multiple sequence alignment algorithm;
a data processing module, configured to use a hidden Markov model to search for amino acid sequence information matched with the signature sequence of the dual-pore structure, and remove redundant data information;
a locating and screening module, configured to locate and screen amino acid sequences obtained from the data processing module to obtain candidate sequences;
a calculation module, configured to calculate a matching length and an envelope length of the candidate sequences; and a registration analysis module, configured to perform registration on the candidate sequences by means of the multiple sequence alignment algorithm, calculate a relative mismatch relationship with the known protein nanopore, and analyze a structure of the candidate sequences to obtain a final sequence.

An embodiment of the present disclosure further provides a system for screening an amino acid sequence of a protein nanopore, including
one or more processors; and
a storage device, configured to store one or more programs, wherein
when one or more programs are executed by one or more processors, the one or more processors implement a screening method for an amino acid sequence of a protein nanopore.

An embodiment of the present disclosure further provides a computer storage medium, on which a computer program is stored. The computer program implements, when being executed by a processor, a screening method for an amino acid sequence of a protein nanopore.

The protein nanopore is a new protein system having two reading units, and has a wide prospect in nanopore single molecule detection, substance structure analysis thereof and other aspects. The screening method for a protein nanopore provided in the present disclosure can screen and obtain protein nanopores with a more novel sequence and structure. A series of amino acid sequences of the protein nanopore obtained by screening have relatively low similarity to the complete sequences and core sequences of type II (T2SS) and type Ill (T3SS) secretin proteins, for example, being obviously different from the amino acid sequences such as CsgG and VcGspD.

The protein nanopores screened in some embodiments of the present disclosure have a longer amino acid in the central gate region and the cap gate region, is newly added with a small segment of helical structure in a key region of the cap gate, has a longer junction fragment in the central region, and is simpler at the N3 terminal.

For the novel protein nanopore and sequences thereof provided in the present disclosure, the sequence homology has relatively low similarity to the sequences disclosed in the prior art; the sequence specific to the protein nanopore reduces the inner diameter of the pore, so that the pore diameter of the channel is relatively small, and protein nanopores formed from certain specific amino acids are merely 5.3 Å, and the sequence thereof changes the charges around the pore, and enhances selectivity of the pore. The nanopore channel protein has a higher isoelectric point, and can be applied in many fields such as substance detection or seawater desalination.

### EXAMPLES

In the following examples, unless otherwise specified, reagents and consumables are purchased from conventional reagent suppliers in the art; and unless otherwise specified, all experimental methods and technical means used are conventional methods and means in the art.

### Example 1 Screening of Amino Acid Sequence of Protein Nanopore

The present example provides a screening method for an amino acid sequence of a protein nanopore. The screening method includes the following steps.

(1) acquiring amino acid information about a known protein nanopore, and obtaining a signature sequence of a dual-pore structure by means of a multiple sequence alignment algorithm.

Firstly, amino acid information about known domain sequences of T2SS and T3SS was searched and obtained from https://wwwscsb.orgisearch; secondly, these amino acid sequences were subjected to a multiple sequence alignment algorithm (MAFFT v7.273) to obtain a template. Signature sequences for a known dual-pore structure are represented by SEQ ID NO.1˜4;

(2) using a hidden Markov model to search for amino acid sequence information matched with the signature sequence of the dual-pore structure, and removing redundant data information.

The amino acid sequence information matched with the dual-pore structure template was searched by the hidden Markov model HMMER v3.3 (possibly also by HmmerWeb v2.41.1); and a parameter used was -E 1--domE 1--incE 0.01--incdomE 0.03--mx BLOSUM62--pextend 0.4--popen 0.02--seqdb uniprotrefprot, wherein uniprotrefprot (v.2019_09) is database information after 100% similarity redundancy removal to UniProtKB (v.2019_09), which can greatly avoid collection of repeated amino acid sequence information.

(3) locating and screening amino acid sequences obtained in step (2) to obtain candidate sequences, and calculating a matching length and an envelope length of the candidate sequences.

(4) performing registration on the candidate sequences by means of the multiple sequence alignment algorithm, calculating a relative mismatch relationship with the known protein nanopore, and analyzing a structure of the candidate sequences to obtain a final sequence.

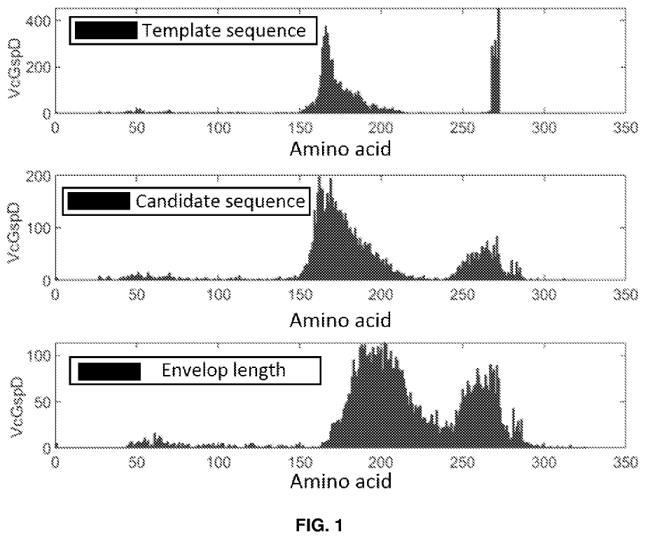

FIG. 1 shows lengths of an initial candidate sequence obtained after searching VcGspD (PDB: 5WQ8) template, in which from top to bottom are a matching length of a template sequence (QUERY) matching part, a length of a candidate sequence (TARGET) matching part, and an envelope length (TARGET ENVELOPE) of the candidate sequence, respectively.

Two conserved matching regions of “KDT” and “LAS” of candidate sequences are located and screened by scripts, and the length of most sequences is more than 150 amino acids, which conforms to a size of a secretin core region, and meanwhile, the sequence length roughly obeys two Gaussian distributions, in which one is similar to the length of the template sequence, and the other is consistent with the length with the S domain or the S+N3 domain removed.

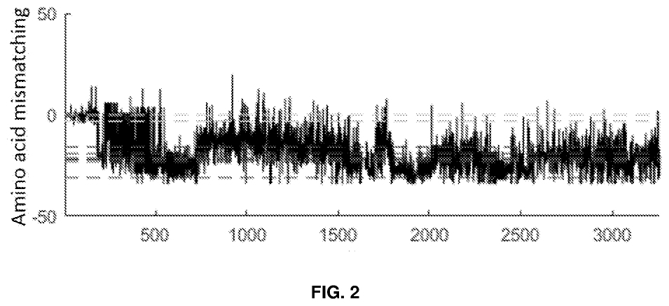

All candidate sequences were registered by means of the multiple sequence alignment algorithm (MAFFT v7.273), and a mismatch relationship relative to VcGspD can be calculated. The mismatch relationship between the candidate sequences and VcGspD is as shown in FIG. 2.

In the above, dotted lines are mismatch values (−4˜0) of 4 known dual-pore structures in Table 1 and mismatch values of known single-pore secretory channels.

Meanwhile, structural analysis was performed on all candidate sequences using MODELLER v10.1 and HOLE2 v2.2.005 with the sequence of the secretin domain of VcGspD as a template.

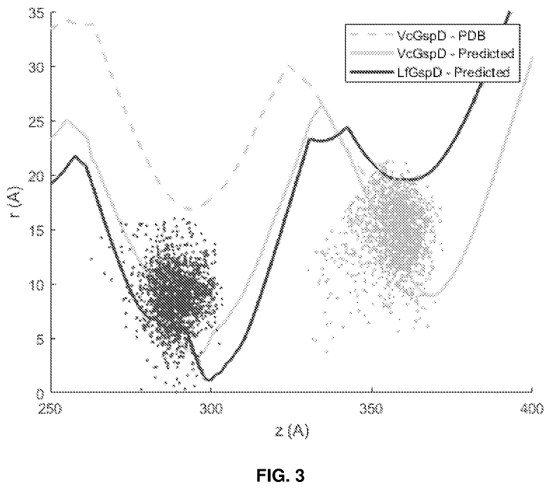

FIG. 3 shows a radial relationship between a filtered sequence and a VcGspD channel, including the size of the channel in VcGspD-PDB, the size after calculation and analysis, and the size after calculation and analysis of candidate sequence LfGspD.

Since the gating region has a switching function, in order to keep the biophysical elasticity in the practical analysis, all of those within a certain circle center radius range were effective values. Left scatters are the central gate region of the candidate sequences, while right scatters are the cap gate region of the candidate sequences, a radius of the latter being slightly larger than that of the former by 5 Å.

The final sequence obtained by the above screening method has a highly controllable central gate narrow channel and cap gate channel. Repetitive sequences identical to the signature sequence of the known dual-pore structure were removed, and representative sequences having 75% similarity were as stated above.

### Example 2 Information Characteristics of C6HW33_9 BACT Protein Nanopore

Homology of an amino acid sequence of a protein nanopore (C6HW33_9 BACT) provided in the present disclosure to the proteins in type II (T2SS) and type III (T3SS) secretion systems that have been reported is shown in the present example.

The amino acid sequence of C6HW33_9 BACT is represented by SEQ ID NO.9.

Reported T2SS protein is found in Korotkov, K. V.; Sandkvist, M.; Hol, W. G. J. The Type II Secretion System: Biogenesis, Molecular Architecture and Mechanism. Nat. Rev. Microbiol. 2012, 10 (5), 336-351. https://doi.org/10.1038/nrmicro2762, and the homology analysis of the protein sequence C6HW33_9 BACT provided in the present disclosure and protein of T2SS is shown in Table 2.

TABLE 2

| | Protein | Query Cover | | | Percent identity (feature similarity) | | |
|---|---|---|---|---|---|---|---|
| Strain | Name | II | III | I | II | III | I |
| Enterotoxigenic Escherichia | GspD | 79% | 73% | 83% | 28.18% | 28.14% | 29.55% |
| coli ETEC | (K12-GspD) | | | | | | |
| Vibrio cholerae | EpsD | 78% | 80% | 79% | 30.03% | 27.84% | 29.22% |
| | (VcGspD) | | | | | | |
| Aeromonas hydrophila | ExeD | 78% | 69% | 79% | 31.68% | 31.88% | 32.18% |
| Dickeya dadantii | OutD | 80% | 68% | 87% | 29.26% | 30.42% | 30.79% |
| (Klebsiella oxytoca) | PulD | 79% | 75% | 89% | 27.69% | 29.13% | 26.93% |
| Pseudomonas aeruginosa | XcpQ | 78% | 79% | 95% | 31.39% | 30.79% | 33.14% |
| Xanthomonas campestris | XpsD | 69% | 55% | 76% | 23.27% | 23.69% | 22.40% |

The reported T3SS protein is found in: Deng, W.; Marshall, N. C.; Rowland, J. L.; McCoy, J. M.; Worrall, L. J.; Santos, A. S.; Strynadka, N. C. J.; Finlay, B. B. Assembly, Structure, Function and Regulation of Type Ill Secretion Systems. Nat. Rev. Microbiol. 2017, 15(6), 323-337. https://doi.org/10.1038/nrmicro. 2017.20, and the homology analysis of the protein sequence C6HW33_9 BACT provided in the present disclosure and protein of T3SS is shown in Table 3.

TABLE 3

| | | Query Cover | | | Percent Identity (feature similarity) | | |
|---|---|---|---|---|---|---|---|
| Strain | Protein | II | III | I | II | III | I |
| Yersinia spp. | YscC | 67% | 55% | 62% | 23.82% | 22.71% | 22.61% |
| Salmonella spp SPI-1 | InvG | 41% | 53% | 59% | 22.67% | 23.96% | 19.87% |
| Salmonella spp SPI-2 | SsaC | 26% | 30% | 30% | 23.33% | 23.12% | 24.36% |
| Enteropathogenic Escherichia coli and | EscC | 78% | 74% | 77% | 23.33% | 23.11% | 22.73% |
| Enterohemorrhagic Escherichia coli | | | | | | | |
| EPEC and EHEC | | | | | | | |
| Shigella spp. | MxiD | 9% | 9% | 16% | 33.96% | 32.08% | 25.00% |
| Chlamydia spp. | CdsC | 77% | 81% | 87% | 25.82% | 26.81% | 28.88% |
| P. aeruginosa | PscC | 67% | 12% | 14% | 23.71% | 33.80% | 34% |
| P. syringae | HrcC | 35% | 33% | 64% | 24.51% | 24.75% | 21.25% |

By analysis, the sequence C6HW33_9 BACT provided in the present disclosure has similarity of less than 40% to the reported functional sequence, and has no similarity to T8SS (CsgG) and RhcC1-RhcC2, etc., and thus is a novel nanopore protein that can be used for nanopore single molecule detection.

### Example 3 Prediction of Structure of Protein Nanopore

The present example was used to predict the structure of a protein nanopore formed from a protein sequence provided in the present disclosure. Structural prediction methods are AlphaFold v2, SWISS-MODEL, RoseTTAFold, Modeller, and I-TASSER.

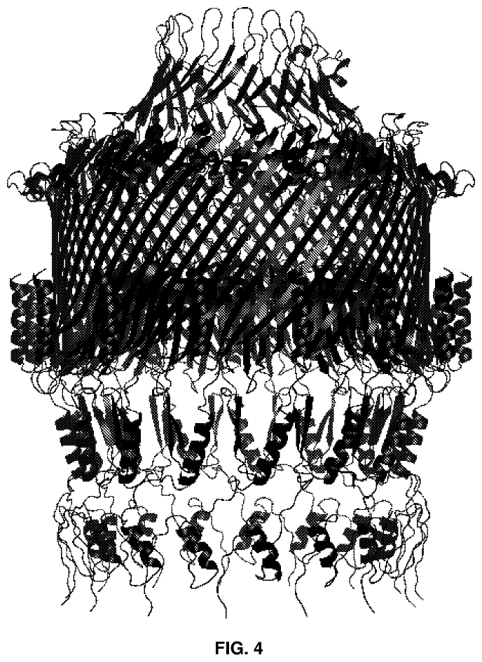

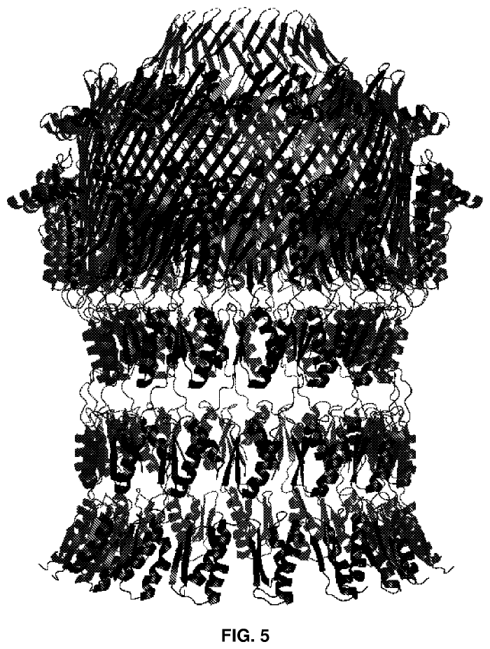

A structure of the protein nanopore C6HW33_9 BACT predicted in the present example is as shown in FIG. 4, compared with the nanopore structure formed from the protein VcGspD in V. cholerae (as shown in FIG. 5).

The protein nanopore sequence provided in the present disclosure is shorter, has 565 amino acids, 119 less than VcGspD, has a higher isoelectric point 9.71 (VcGspD has an isoelectric point 4.8), and has longer cap gate and central gate amino acid sequences.

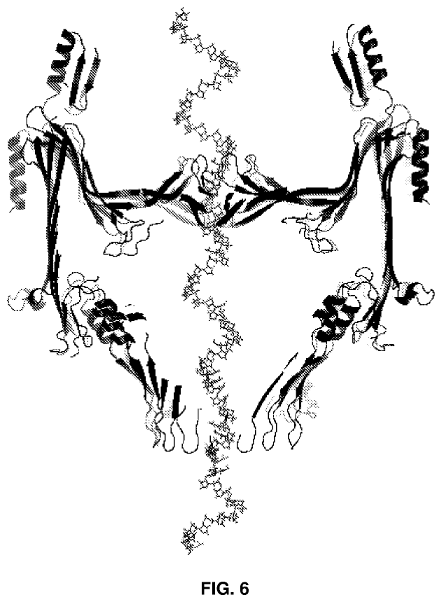

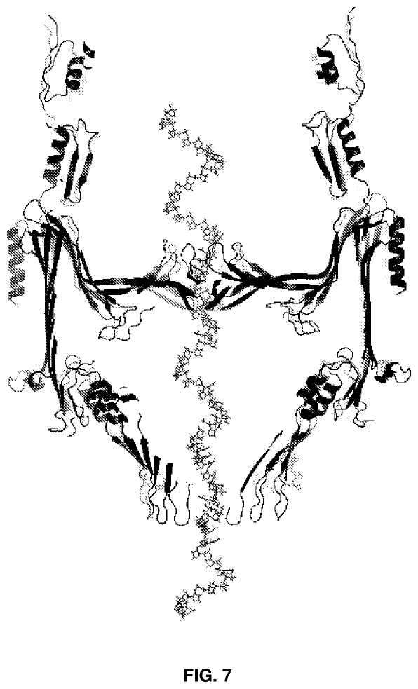

In addition, FIG. 6 is a schematic diagram of nucleic acid translocation through a mutant protein nanopore, and FIG. 7 shows a single molecule nucleic acid crossing a wild-type protein nanopore.

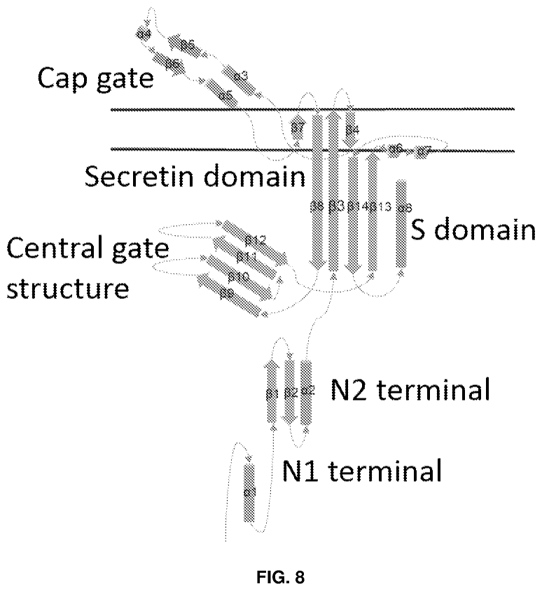

The protein structure predicted in the present example shows (as in FIG. 8) that the protein nanopore is newly added with a small segment of helical structure in the cap gate region, and has a longer junction fragment in the central gate region.

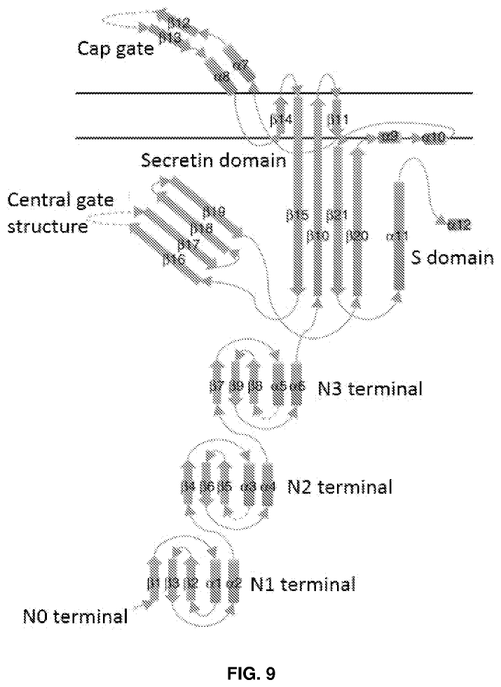

Besides, compared with interaction between N3 terminal and S region via a hydrogen bond in VcGspD (FIG. 9), the monomeric protein in the present disclosure is much simpler at N3 terminal.

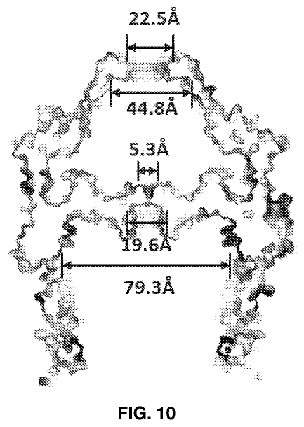

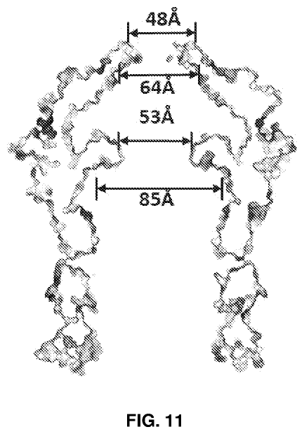

According to analysis with SWISS-model, the protein provided in the present disclosure can form a nanopore structure, wherein in the naturally formed 15-mer nanopore structure, as shown in FIG. 10, a pore channel thereof is only 5.3 Å, much smaller than that of VcGspD (FIG. 11) and protein nanopore structures having been reported currently.

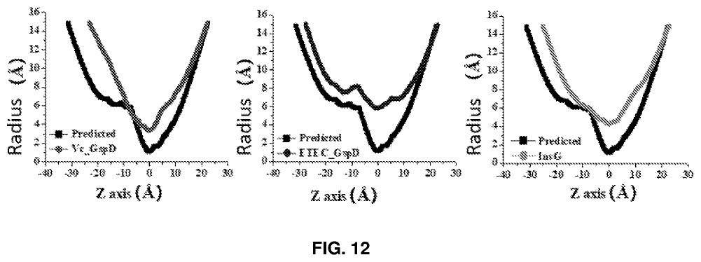

In addition, FIG. 12 shows pore diameters of protein nanopores of VcGspD, ETEC_GspD, and InvG.

### Example 4 Structural Simulation of Proteins

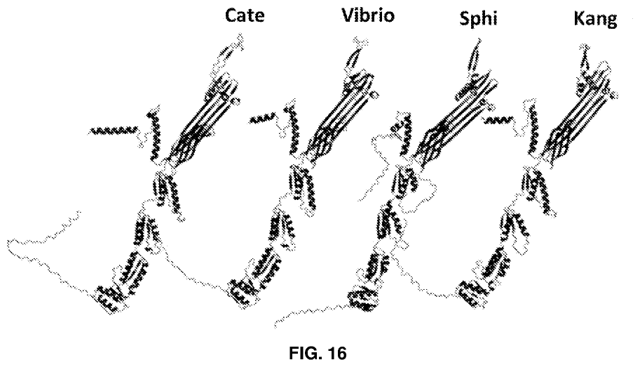

The present disclosure predicted structures of four proteins U3AQV9_9 VIBR (Vibrio azureus), A0A0J8GPG7_9 ALTE (Catenovulum maritimum), C7R8G0_KANKD (Kangiella koreensis) and A0A0E9M078_9 SPHN (Sphingomonas changbaiensis NBRC 104936) using Hermite and the protein nanopore structure prediction method in Example 3 (AlphaFold v2). The predicted proteins all have a cap gate region, as shown in FIG. 16.

In the present disclosure, protein nanopores obtained from the screening meanwhile were randomly selected, including: A0A2R4XIB8_9 BORD, U4KHA5_9 VIBR, D4ZEB1 SHEVD, A0A1M5Z8V4_9GAMM, K7AHG1_9ALTE, A3WP11_9GAMM, C6XJ47 HIRBI, G4E4N3_9GAMM, N9BSP8_9GAMM, GOAE23 COLFT, A0A3N8KT41_9BURK, B9TP47 RICCO, H5WJ69_9BURK, A0A1P8WL02_9PLAN, M5TB48_9PLAN, Q221 L0 RHOFT, etc. Characteristics of the obtained protein nanopores are similar to those of C6HW33_9 BACT. Since there are many amino acid sequences obtained from the screening, the present patent only shows C6HW33_9 BACT, U3AQV9_9 VIBR, A0A0J8GPG7_9ALTE, C7R8G0 KANKD, and A0AOE9MQ78_9 SPHN as representatives, avoiding redundant description.

### Example 5 Mutation Modification of Protein Nanopore

Taking C6HW33_9 BACT as an example, mutants were designed for obtained sequences as follows, and mutants and mutant effects obtained are as shown in Table 4 below:

TABLE 4

| Protein Mutant | Mutation Position | Effect |
|---|---|---|
| K441A/R442Q | Central gate charge | Reinforcing central gate |
| | removal | |
| Del (N1-V185) | Nitrogen terminal | Removing nitrogen |
| | deletion | terminal |
| Del (S262-G322) | Cap gate deletion | Removing cap gate |
| Del (K364-T403, | Central gate mutation | Removing central gate |
| V416-T447) | | |
| K441A/R442Q, Del | Cap gate deletion and | Removing cap gate |
| | central gate mutation | pore and reinforcing |
| | | central pore |
| Del (S382-N386) | Central gate mutation | Enlarging central pore |
| S284G, S308G | Cap gate mutation | Reducing cap gate size |

As can be seen from the above table, after the sequence is subjected to point mutation modification, a structure of a protein nanopore obtained and functions of various amino acid residues are clearer, providing a research basis for subsequent modification and application of the protein nanopore.

### Example 6 Protein Nanopore Expression and Purification Methods

Taking C6HW33_9 BACT as an example, a gene encoding a protein nanopore was synthesized, a histidine tag and a polypeptide enzymatic protease sequence were added to N-terminal of the gene, transformed into E. coli C43 expression strains, and screened on an agar plate containing 100 μg/mL antibiotics to obtain single colonies.

The single colonies were picked up, cultured at 37° C. under a condition of 200 rpm until OD was greater than 1.2, and subjected to enlarged culture at 1:200 (seed solution/culture medium). When OD₆₀₀ was greater than 0.6, IPTG was added, temperature was lowered to below 16° C., and culturing was continued for more than 14 h. Thalli were collected by 4000 g, and washed once with a phosphate buffer solution with pH 7.4.

150 mM NaCl, 15 mM Tris-HCl, 1 mM imidazole, 0.5 mM PMSF, and 25 Wml nuclease were added at a weight-to-volume ratio of 1:10.

Then cells were lysed by ultrasonication (turning on for 1 s, turning off for 2 s, for 40 min), cell debris were removed by 4000 g, 0.2% amphiphilic detergent Zw3-14 was added, mixture was well mixed on ice for 1 h, filtered with a 0.22 μm filter to obtain supernatant, and then the supernatant was injected into a Ni agarose column.

Resultant was washed with a solution A (150 mM NaCl, 15 mM Tris-HCl, 1 mM imidazole, 0.2% Zw3-14), a solution B (150 mM NaCl, 15 mM Tris-HCl, 20 mM imidazole, 0.2% Zw3-14), and a solution C (150 mM NaCl, 15 mM Tris-HCl, 50 mM imidazole, 0.2% Zw3-14) in sequence, an eluent (150 mM NaCl, 15 mM Tris-HCl, 500 mM imidazole, 0.2% Zw3-14) was added to collect protein.

The collected protein was further subjected to polymer and monomer separation by gel chromatographic molecular sieve, where an elution liquid was 150 mM NaCl, 15 mM Tris-HCl, and 0.2% Zw3-14.

### Example 7 Electrophysiological Characterization of C6HW33_9 BACT Protein Nanopore

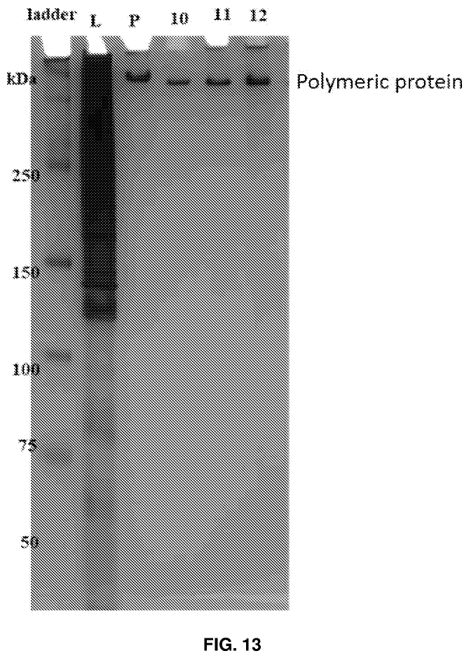

C6HW33_9 BACT protein nanopores were expressed by the method of Example 5. Results of SDS-PAGE electrophoresis in combination with silver staining of the protein obtained by purification are shown in FIG. 13. The protein obtained by purification was stored in a buffer of 150 mM NaCl, 15 mM Tris-HCl, and 0.1% DDM.

The protein obtained by purification was further separated by Blue-native PAGE, and a polymer strip thereof was gel-cut, and extracted with the above liquid. To a 100 μm of biochip, 150 μl of a solution of 300 mM NaCl and 20 mM HEPES with pH 7.5 was added. A layer of phospholipid was coated to form a lipid bilayer. The protein recovered from gel-cutting was added to form a transmembrane channel.

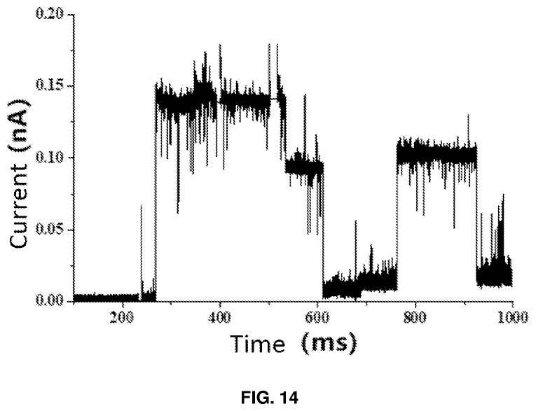

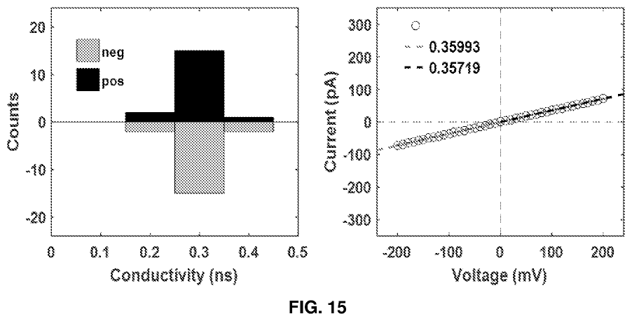

After a single molecule transmembrane channel was obtained, an electrical signal was recorded by electrophysiological instrument, and a result is as shown in FIG. 14, in which secondary transition exists in the current, and the white cap gate and central gate simultaneously responded to the current signal. The current of the C6HW33_9 BACT protein single molecule channel was analyzed under different voltages (−200 mV˜200 mV), and a resistivity was calculated by linear fitting. Results are as shown in FIG. 15, in which the current of the protein changes linearly under a voltage of −200 mV-200 mV, and the resistivity is 0.35 nS.

### Example 8 Electrophysiology of Vibrio, Cate, Kang, and Sphi of Protein Nanopore

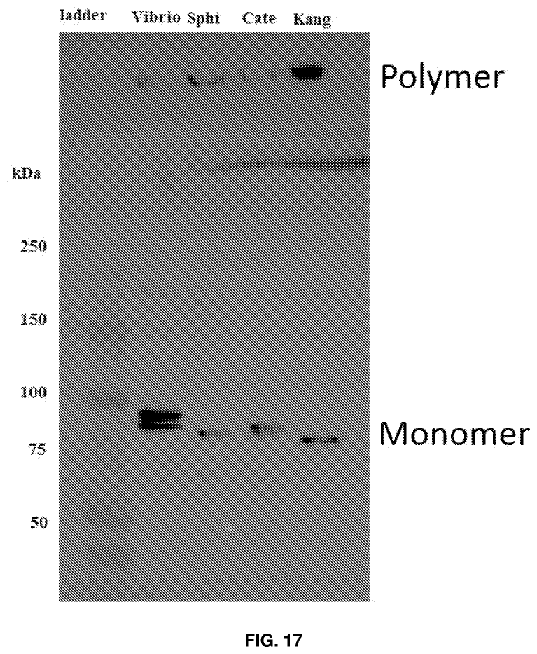

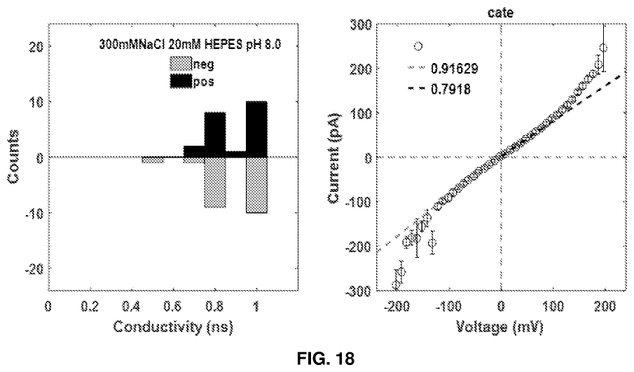

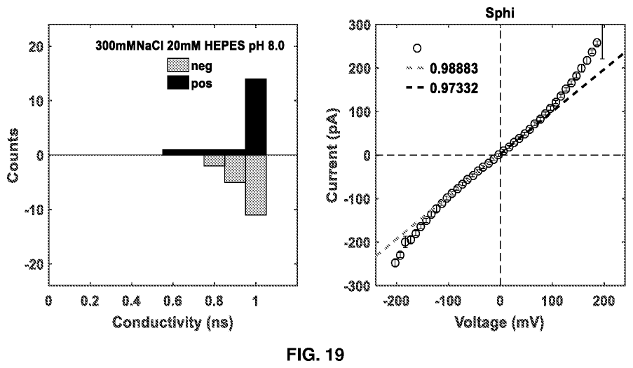

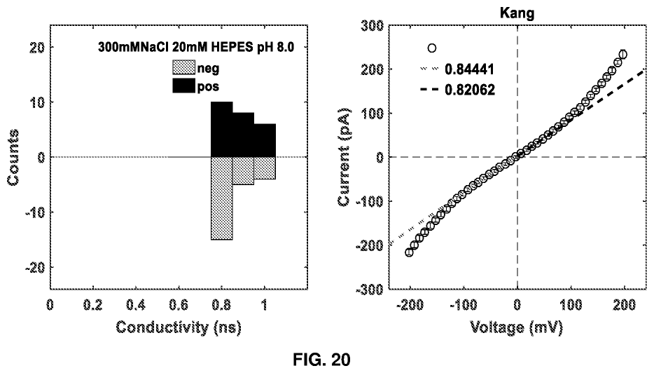

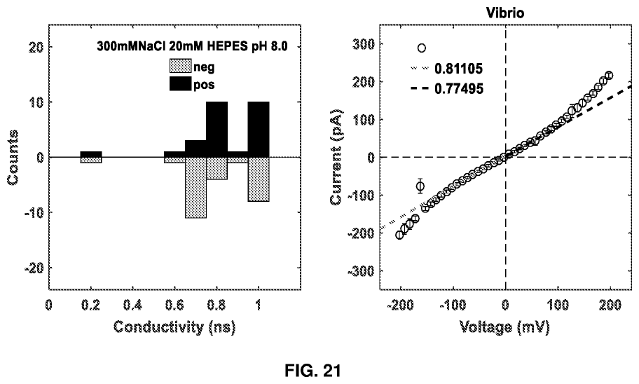

Proteins of U3AQV9_9VIBR (Vibrio azureus), A0A0J8G PG 7_9ALTE (Catenovulum maritimum), C7R8G0_KANKD (Kangiella koreensis), and A0AOE9MQ78_9 SPHN (Sphingomonas changbaiensis NBRC 104936)) were purified and obtained by the method of Example 5. Proteins and polymers of the four proteins were detected by immunoblotting, as shown in FIG. 17. Electrophysiology of the four proteins was detected by the method of Example 7, and results are shown in FIGS. 18 to 21 respectively. In a solution environment of 300 mM NaCl, 20 mM HEPES, and pH 7.5, the currents of the four proteins change linearly under a voltage of −200 mV-200 mV, and the resistivity is 0.7 nS-1 nS.

In conclusion, by the screening method for the protein nanopore in the present disclosure, a series of amino acid sequences of the protein nanopore screened have relatively low similarity to the complete sequences and the core sequences of the type II (T2SS) and type III (T3SS) secretin proteins, and have the central gating region and the cap gate region sequences in structure, wherein a part of the protein nanopores have longer amino acid sequences in the cap gate region and the central gate region. Functionally, the special cap gate and central gate sequences of the protein nanopores in the present disclosure constitute a smaller channel, which reduces the resistivity of the pore channel, and enhances resolving ability of the pore to translocation of substance through the pore. The special sequences change the charges around the pore, and enhance the selectivity of the pore. The protein nanopore in the present disclosure can be applied to many fields such as substance detection and seawater desalination.

The applicant declares that the above-mentioned are merely embodiments of the present disclosure, but the scope of protection of the present disclosure is not limited thereto. All parameters, sizes, materials, and configurations described herein are exemplary. Those skilled in the art would know that any variation or substitution readily conceivable to those skilled in the art based on the present disclosure in the technical scope disclosed in the present disclosure falls within the scope of protection of the present disclosure and the disclosure scope.

### INDUSTRIAL APPLICABILITY

The present disclosure provides a screening method for an amino acid sequence of a protein nanopore, a protein nanopore, and applications thereof. The protein nanopore formed from the amino acid sequence screened by the method has relatively low similarity to the known secretin proteins of T2SS, T3SS, and T4SS. The protein nanopore has central gate and cap gate structures, so that a channel thereof has a small pore diameter and high selectivity. The sequence specific to both the central gate region and the cap gate region reduces the inner diameter of the pore, improves the resolving ability of the pore channel. The protein nanopore in the present disclosure is a novel type of protein nanopore with good selectivity, can be applied to many fields such as substance detection or seawater desalination, has excellent practical performance, and can be widely applied to the field of electrical and/or optical signal detection of an object to be detected.

What is claimed is:

1. A screening method for an amino acid sequence by a protein nanopore, wherein the screening method comprises following steps in sequence:
(1) acquiring amino acid information about a known protein nanopore, and evaluating a signature sequence of a dual-pore structure by a multiple sequence alignment algorithm;
(2) using a hidden Markov model to search for amino acid sequence information matched with the signature sequence of the dual-pore structure, and removing redundant data information;
(3) locating and screening amino acid sequences obtained in step (2) to obtain candidate sequences, and calculating a matching length and an envelope length of the candidate sequences; and
(4) performing registration on the candidate sequences by the multiple sequence alignment algorithm, calculating a relative mismatch relationship with the known protein nanopore, and analyzing a structure of the candidate sequences to obtain a final sequence.

2. The screening method according to claim 1, wherein the signature sequence of the dual-pore structure in step (1) is any one of amino acid sequences represented by protein SEQ ID NO.1˜4.

3. A protein nanopore, wherein the protein nanopore contains cap gate and central gate structures; and an amino acid sequence of the protein nanopore is any one of amino acid sequences screened by the screening method according to claim 1.

4. The protein nanopore according to claim 3, wherein the protein nanopore is a polymer composed of monomeric proteins of any one of the amino acid sequences.

5. The protein nanopore according to claim 3, wherein the protein nanopore contains a central gate signature sequence or a cap gate signature sequence or an isoelectric point determination sequence.

6. The protein nanopore according to claim 3, wherein the protein nanopore contains a modification structure.

7. A single-pore protein nanopore, wherein the single-pore protein nanopore is obtained in a following manner: making one or more deletions to S262-G322 segment of the protein nanopore according to claim 3, and removing a cap gate region.

8. The screening method according to claim 2, wherein conserved match regions used for locating and screening the candidate sequences in step (3) are KDT and LAS.

9. The screening method according to claim 2, wherein the final sequence in step (4) has similarity of 75% or less to the known protein nanopore.

10. The screening method according to claim 2, wherein amino acids screened by the screening method are as shown in a following Table 1:
TABLE 1 [symbol] [symbol] [symbol] [symbol] [symbol] [symbol]
. [symbol]
indicates data missing or illegible when filed

11. The protein nanopore according to claim 4, wherein the polymer comprises 12˜16-mers.

12. The protein nanopore according to claim 5, wherein the isoelectric point determination sequence is an amino acid sequence represented by SEQ ID NO.5 or a sequence having homology greater than 75% to SEQ ID NO.5, wherein
the SEQ ID NO.5 sequence is:
KAKITVGEDVPFITGQSQTVGGNVMTMIQRQNVGIT.

13. The protein nanopore according to claim 5, wherein the cap gate signature sequence is an amino acid sequence represented by SEQ ID NO.6, SEQ ID NO.10, SEQ ID NO.11 or SEQ ID NO.12, or a sequence having homology greater than 75% to SEQ ID NO.6, SEQ ID NO.10, SEQ ID NO.11 or SEQ ID NO.12, wherein
the SEQ ID NO.6 sequence is:
GATGASSLSGSTTGAAGSLGVVSGAAGAASALSG,
the SEQ ID NO.10 sequence is:
RTRKEPDDITYRTDAAGQPIYNNNGNRVIASITEGKEIQGDFG,
the SEQ ID NO.11 sequence is:
GPRNVATVPLGQDLTQPPVAGTG,
and
the SEQ ID NO.12 sequence is:
GNIVVDANGNAVTQTTSTQGDFTALASLLGGLNG.

14. The protein nanopore according to claim 5, wherein the central gate signature sequence is an amino acid sequence represented by SEQ ID NO.7, SEQ ID NO.8, SEQ ID NO.13, SEQ ID NO.14 or SEQ ID NO.15, or a sequence having homology greater than 75% to SEQ ID NO.7, SEQ ID NO.8, SEQ ID NO.13, SEQ ID NO.14 or SEQ ID NO.15, wherein
the SEQ ID NO.7 sequence is:
QSQTVGGNVMTMIQ;
the SEQ ID NO.8 sequence is:
QTITALTNASQLIGTMAVGPTTT,
the SEQ ID NO.13 sequence is:
PTITGATASTNNTNPFQTVERK,
the SEQ ID NO.14 sequence is:
QVPILQALAAGNAAFQNVTY,
and
the SEQ ID NO.15 sequence is:
PILTGTTASAGSSNPATTVDRQ.

15. The protein nanopore according to claim 6, wherein positions modified by the modification structure comprise a central gate, a cap gate, N-terminal, or C-terminal.

16. The protein nanopore according to claim 6, wherein modification of the modification structure comprises at least one of: 1) adding at least one amino acid or unnatural amino acid, 2) reducing at least one amino acid; and 3) performing substituting or modifying on a side chain on at least one amino acid in the modification structure.
